SEQUENCE LISTING (34 sequences)

SEQ ID NO: 1 - protein, 100 aa, synthetic construct
GDFVKPGSLSVKVTDWGNTEYDVTLNLGGTYDWVVKVKLKDGSSVSSFWSANKAEEGGYVVFTPVSWNRGPTATFGFIATGSESVEAIYLYVDGQLWDAW

SEQ ID NO: 2 - DNA, 300 bp, synthetic construct
  source 1..300: mol_type = other DNA
ggtgatttcgttaaaccgggcagcctgagcgttaaagttaccgattggggcaacaccgaatacgatgttaccctgaacctgggcggcacctacgattgggttgttaaagttaaactgaaagatggcagcagcgttagcagcttctggagcgcgaacaaagcggaagaaggcggctacgttgttttcaccccggtttcttggaaccgtggtccgaccgcgaccttcggtttcatcgcgaccggttctgaaagcgttgaagcgatctacctgtacgttgatggtcagctgtgggatgcgtgg

SEQ ID NO: 3 - protein, 104 aa, synthetic construct
GSGDLVKPDAFSVKIQDWGSTEYDVTLNLGGTYDWVVKVKLKDGSAVSSVWSANKAEEGGYVVFTPVSWNKGPTATFGFIATGSEPVEAMYLYVNDQLWDVWLE

SEQ ID NO: 4 - DNA, 312 bp, synthetic construct
  source 1..312: mol_type = other DNA
ggatccggtgatctggttaaaccggatgcgttcagcgttaaaatccaggattggggcagcaccgaatacgatgttaccctgaacctgggcggcacctacgattgggttgttaaagttaaactgaaagatggcagcgcggttagcagcgtttggagcgcgaacaaagcggaagaaggcggctacgttgttttcaccccggttagctggaacaaaggcccgaccgcgaccttcggcttcatcgcgaccggcagcgaaccggttgaagcgatgtacctgtacgttaacgatcagctgtgggatgtttggctcgag

SEQ ID NO: 5 - protein, 166 aa, synthetic construct
HGYVASPGSRAFFGSSAGGNLNTNVGRAQWEPQSIEAPKNTFITGKLASAGVSGFEPLDEQTATRWHKTNITTGPLDITWNLTAQHRTASWDYYITKNGWNPNQPLDIKNFDKIASIDGKQEVPNKVVKQTINIPTDRKGYHVIYAVWGIGDTVNAFYQAIDVNIQ

SEQ ID NO: 6 - DNA, 498 bp, synthetic construct
  source 1..498: mol_type = other DNA
cacggttacgttgcttctccgggtagccgtgcgttcttcggcagctctgcgggcggtaacctgaacaccaacgttggccgtgcgcagtgggaaccgcagagcatcgaagcgccgaaaaacaccttcatcaccggtaaactggcgagcgcgggcgttagcggtttcgaaccgctggatgaacagaccgcgacccgttggcacaaaaccaacatcaccaccggcccgctggatatcacctggaacctgaccgcgcagcaccgtaccgcgagctgggattactacatcaccaaaaacggctggaacccgaaccagccgctggatatcaaaaacttcgataaaatcgcgagcatcgatggcaaacaggaagttccgaacaaagttgttaaacagaccatcaacatcccgaccgatcgtaaaggctaccacgttatctacgcggtgtggggcattggtgacaccgttaacgcattttatcaggcgatcgacgttaacatccag

SEQ ID NO: 7 - protein, 358 aa, synthetic construct
GSLSNQSGYPAFRGLMSWSINWDAKNNFEFSNNYRTYFDGLSLQKGPNANPIPEHFFAPYIDMSLSVHKPLVEYAKLTGTKYFTLAFILYSSVYNGPAWAGSIPLEKFVDEVRELREIGGEVIIAFGGAVGPYLCQQASTPEQLAEWYIKVIDTYNATYLDFDIAAGIDADKLADALLIVQRERPWVKFSFTLPSDPGIGLAGGYGIIETMAKKGVRVDRVNPMTMDYYWTPSNAENAIKVAENVFRQLKQIYPEKSDEEIWKMIGLTPMIGVNDDKSVFTLEDAQQLVDWAIQHKIGSLAFWSVDRDHPGPTGEVSPLHRGTNDPDWAFSHVFVKFMEAFGYTFSAQTSEASVPTLE

SEQ ID NO: 8 - DNA, 1074 bp, synthetic construct
  source 1..1074: mol_type = other DNA
ggatccctgagcaaccagagcggctacccggcgttccgtggcctgatgagctggtccatcaactgggatgcgaaaaacaacttcgaattcagcaacaactaccgtacctacttcgatggtctgagcctgcagaaaggcccgaacgcgaacccgatcccggaacacttcttcgcgccgtacatcgatatgtctctgtctgttcacaaaccgctggttgaatacgctaaactgaccggtaccaaatacttcaccctggcgttcatcctgtactccagcgtttacaacggcccggcgtgggcgggtagcatcccgctggaaaaattcgttgacgaagtgcgtgaactgcgtgaaatcggtggtgaagtgatcatcgcgttcggcggcgcggttggtccgtacctgtgccagcaggcgagcaccccggaacagctggcggaatggtacatcaaagttatcgacacctacaacgctacctacctggattttgatattgcggcgggtatcgatgctgacaaactggcggatgctctgctgattgttcagcgtgaacgtccgtgggttaaattctctttcaccctgccgagcgatccgggcatcggcctggcaggtggttacggtatcatcgaaaccatggcgaaaaaaggcgttcgtgttgatcgtgttaacccgatgaccatggactactattggaccccgtctaacgctgaaaacgcgattaaagttgcggaaaacgttttccgtcagctgaaacagatctacccggaaaaatctgatgaagaaatctggaaaatgatcggtctgaccccgatgatcggtgttaacgatgacaaatctgtgttcaccctggaagatgctcagcagctggttgattgggcgatccagcacaaaattggctctctggcgttctggagcgttgatcgtgatcacccaggcccgaccggtgaagtttctccgctgcaccgtggcaccaacgatccggattgggctttcagccacgttttcgttaaattcatggaagcattcggctacaccttcagcgcgcagacctctgaagcgtctgttccgaccctcgag

SEQ ID NO: 9 - protein, 104 aa, synthetic construct
GPTTPVPVSGSLEVKVNDWGSGAEYDVTLNLDGQYDWTVKVKLAPGATVGSFWSANKQEGNGYVIFTPVSWNKGPTATFGFIVNGPQGDKVEEITLEINGQVIL

SEQ ID NO: 10 - DNA, 234 bp, synthetic construct
  source 1..234: mol_type = other DNA
gatgttaccctgaacctggatggtcagtacgattggaccgttaaagttaaactggcgccgggtgcgaccgttggtagcttctggtctgcgaacaaacaggaaggtaacggttacgttatcttcaccccggtttcttggaacaaaggtccgaccgcgaccttcggtttcatcgttaacggtccgcagggtgataaagttgaagaaatcaccctggaaatcaacggtcaggttatc

SEQ ID NO: 11 - protein, 333 aa, synthetic construct
ANNLGSKLLVGYWHNFDNGTGIIKLKDVSPKWDVINVSFGETGGDRSTVEFSPVYGTDADFKSDISYLKSKGKKVVLSIGGQNGVVLLPDNAAKDRFINSIQSLIDKYGFDGIDIDLESGIYLNGNDTNFKNPTTPQIVNLISAIRTISDHYGPDFLLSMAPETAYVQGGYSAYGSIWGAYLPIIYGVKDKLTYIHVQHYNAGSGIGMDGNNYNQGTADYEVAMADMLLHGFPVGGNANNIFPALRSDQVMIGLPAAPAAAPSGGYISPTEMKKALNYIIKGVPFGGKYKLSNQSGYPAFRGLMSWSINWDAKNNFEFSNNYRTYFDGLSLQK

SEQ ID NO: 12 - DNA, 999 bp, synthetic construct
  source 1..999: mol_type = other DNA
gccaacaacctgggttctaaactgctggttggttactggcacaacttcgataacggtaccggtatcatcaaactgaaagatgttagcccgaaatgggatgttatcaacgttagcttcggtgaaaccggtggtgatcgttctaccgttgaattcagcccggtttacggtaccgatgcggatttcaaatccgatatcagctacctgaaaagcaaaggtaaaaaagttgttctgtctatcggcggtcagaacggcgttgttctgctgccggataacgcggcgaaagatcgtttcatcaacagcatccagtctctgatcgataaatacggtttcgatggtatcgatatcgatctggaaagcggtatctacctgaacggtaacgataccaacttcaaaaacccgaccaccccgcagatcgttaacctgatcagcgcaatccgtaccatctctgatcactacggcccggatttcctgctgtctatggcgccggaaaccgcgtacgttcagggtggttacagcgcgtacggttctatctggggtgcgtacctgccgatcatctacggtgttaaagataaactgacctacatccacgttcagcactacaacgctggtagcggtatcggcatggatggtaacaactacaaccagggtaccgcggattacgaagttgcgatggcggatatgctgctgcacggcttcccggttggcggtaacgcgaacaacatcttcccggcgctgcgtagcgatcaggttatgatcggtctgccggcggcgccggctgcggctccgagcggtggctacatcagcccgaccgaaatgaaaaaagcgctgaactacatcatcaaaggcgttccgttcggtggcaaatacaaactgagcaaccagagcggctacccggcgttccgtggcctgatgagctggtccatcaactgggatgcgaaaaacaacttcgaattcagcaacaactaccgtacctacttcgatggtctgagcctgcagaaa

SEQ ID NO: 13 - protein, 105 aa, synthetic construct
ATSATATFAKTSDWGTGFGGSWTVKNTGTTSLSSWTVEWDFPTGTKVTSAWDATVTNSGDHWTAKNVGWNGTLAPGASVSFGFNGSGPGSPSNCKLNGGSCDGTS

SEQ ID NO: 14 - DNA, 315 bp, synthetic construct
  source 1..315: mol_type = other DNA
gcgaccagcgcgaccgcgaccttcgcgaaaaccagcgattggggcaccggcttcggcggcagctggaccgttaaaaacaccggcaccaccagcctgagcagctggaccgttgaatgggatttcccgaccggcaccaaagttaccagcgcgtgggatgcgaccgttaccaacagcggcgatcactggaccgcgaaaaacgttggctggaacggcaccctggcgccgggcgcgagcgttagcttcggttttaacggtagcggtccgggttctccgtctaactgtaaactgaacggtggttcttgtgatggtacctct

SEQ ID NO: 15 - RNA, 42 nt, synthetic construct
  source 1..42: mol_type = other RNA
taatttctactaagtgtagatcatagattataaatggcatgg

SEQ ID NO: 16 - DNA, 3684 bp, synthetic construct
  source 1..3684: mol_type = other DNA
gcagcaagtaaactggaaaaatttaccaattgttatagtctgagcaaaacactgcgttttaaagcaattccggttggtaaaacccaggaaaatattgataataaacgcctgctggttgaagatgaaaaacgtgcagaagattataaaggtgtgaaaaagctgctggatcgttattatctgagttttattaacgatgtgctgcatagcattaaactgaaaaacctgaataactatatcagcctgtttcgtaaaaagacccgtaccgaaaaagaaaataaagaactggaaaacctggaaattaacctgcgcaaagaaatcgccaaagcatttaaaggcgccgcaggctataaaagcctgtttaaaaaggatattatcgaaacaatcctgccggaagcagcagatgataaagatgaaatcgcactggtgaatagctttaatggctttaccacagcatttaccggtttttttgataatcgtgaaaacatgtttagtgaagaagcaaaaagcaccagcattgcatttcgctgtattaacgaaaatctgacccgctatattagcaatatggatatttttgaaaaagttgatgcaatctttgataaacatgaagttcaggaaatcaaagaaaaaattctgaatagcgattatgatgttgaagatttttttgaaggtgaattttttaattttgttctgacccaggaaggtattgatgtttataatgcaattattggtggttttgttaccgaaagcggtgaaaaaattaaaggtctgaatgaatatattaatctgtataatgcaaaaaccaaacaggcactgccgaaatttaaaccgctgtataaacaggttctgagcgatcgtgaaagcctgagcttttatggtgaaggttataccagcgatgaagaagttctggaagtttttcgtaataccctgaataaaaatagcgaaatttttagcagcattaaaaaactggaaaaactgtttaaaaattttgatgaatatagcagcgcaggtatttttgttaaaaatggtccggcaattagcaccattagcaaagatatttttggtgaatggaatctgattcgtgataaatggaatgcagaatatgatgatattcatctgaaaaaaaaagcagttgttaccgaaaaatatgaagatgatcgtcgtaaaagttttaaaaaaattggtagctttagcctggaacagctgcaggaatatgcagatgcagatctgagcgttgttgaaaaactgaaagaaattattattcagaaagttgatgaaatttataaagtttatggtagcagcgaaaaactgtttgatgcagattttgttctggaaaaaagcctgaaaaaaaatgatgcagttgttgcaattatgaaagatctgctggatagcgttaaaagttttgaaaattatattaaagcattttttggtgaaggtaaagaaaccaatcgtgatgaaagtttttatggtgattttgttctggcatacgatattctgctgaaagttgatcatatttatgatgcaattcgtaattatgttacccagaaaccgtatagcaaagataaatttaaactgtattttcagaatccgcagtttatgggtggttgggataaagataaagaaaccgattatcgtgcaaccattctgcgttatggtagcaaatattatctggcaattatggataaaaaatatgcaaaatgtctgcagaaaattgataaagatgatgttaatggtaattatgaaaaaattaattataaactgctgccgggtccgaataaaatgctgccgaaagttttttttagcaaaaaatggatggcatattataatccgagcgaagatattcagaaaatttataaaaatggtacatttaaaaaaggtgatatgtttaatctgaatgattgtcataaactgattgatttttttaaagatagcattagccgttatccgaaatggagcaatgcatacgattttaattttagcgaaaccgaaaaatataaagatattgcaggtttttatcgtgaagttgaagaacagggttataaagttagctttgaaagcgcaagcaaaaaagaagttgataaactggttgaagaaggtaaactgtatatgtttcagatttataataaagattttagcgataaaagccacggtacaccgaatctgcataccatgtattttaaactgctgtttgatgaaaataatcatggtcagattcgtctgagcggtggtgcagaactgtttatgcgtcgtgcaagcctgaaaaaagaagaactggttgttcatccggcaaatagcccgattgcaaataaaaatccggataatccgaaaaaaaccaccaccctgagctatgatgtttataaagataaacgttttagcgaagatcagtatgaactgcatattccgattgcaattaataaatgtccgaaaaatatttttaaaattaataccgaagttcgtgttctgctgaaacatgatgataatccgtatgttattggtattgatcgtggtgaacgtaatctgctgtatattgttgttgttgatggtaaaggtaatattgttgaacagtatagcctgaatgaaattattaataattttaatggtattcgtattaaaaccgattatcatagcctgctggataaaaaagaaaaagaacgttttgaagcacgtcagaattggaccagcattgaaaatattaaagaactgaaagcaggttatattagccaggttgttcataaaatttgtgaactggttgaaaaatatgatgcagttattgcactggaagatctgaatagcggttttaaaaatagccgtgttaaagttgaaaaacaggtttatcagaaatttgaaaaaatgctgattgataaactgaattatatggttgataaaaaaagcaatccgtgtgcaaccggtggtgcactgaaaggttatcagattaccaataaatttgaaagttttaaaagcatgagcacccagaatggttttattttttatattccggcatggctgaccagcaaaattgatccgagcaccggttttgttaatctgctgaaaaccaaatataccagcattgcagatagcaaaaaatttattagcagctttgatcgtattatgtatgttccggaagaagatctgtttgaatttgcactggattataaaaattttagccgtaccgatgcagattatattaaaaaatggaaactgtatagctatggtaatcgtattcgtatttttgcagcagcaaaaaaaaataatgtttttgcatgggaagaagtttgtctgaccagcgcatataaagaactgtttaataaatatggtattaattatcagcagggtgatattcgtgcactgctgtgtgaacagagcgataaagcattttatagcagctttatggcactgatgagcctgatgctgcagatgcgtaatagcattaccggtcgtaccgatgttgattttctgattagcccggttaaaaatagcgatggtattttttatgatagccgtaattatgaagcacaggaaaatgcaattctgccgaaaaatgcagatgcaaatggtgcatataatattgcacgtaaagttctgtgggcaattggtcagtttaaaaaagcagaagatgaaaaactggataaagttaaaattgcaattagcaataaagaatggctggaatatgcacagaccagcgttaaa

SEQ ID NO: 17 - protein, 269 aa, synthetic construct
GSATCATAWSSSSVYTNGGTVSYNGRNYTAKWWTQNERPGTSDVWADKGACGTGGEGPGGNNGFVVSEAQFNQMFPNRNAFYTYKGLTDALSAYPAFAKTGSDEVKKREAAAFLANVSHETGGLFYIKEVNEANYPHYCDTTQSYGCPAGQAAYYGRGPIQLSWNFNYKAAGDALGINLLANPYLVEQDPAVAWKTGLWYWNSQNGPGTMTPHNAIVNNAGFGETIRSINGALECNGGNPAQVQSRINKFTQFTQILGTTTGPNLSCLE

SEQ ID NO: 18 - DNA, 807 bp, synthetic construct
  source 1..807: mol_type = other DNA
ggatccgccacctgcgcgaccgcgtggagcagctccagcgtttacaccaacggcggcaccgttagctacaacggccgtaactacaccgcgaaatggtggacccagaacgaacgtccgggcacctctgatgtttgggcggacaaaggtgcgtgcggcaccggcggcgaaggtccgggtggcaacaacggtttcgttgttagcgaagcacagttcaaccagatgttcccgaaccgcaacgcgttctacacctacaaaggcctgaccgatgcgctgagcgcgtacccggcgttcgcgaaaaccggtagcgatgaagttaaaaaacgtgaagcggctgcgttcctggcgaacgtgtcccacgaaaccggtggcctgttctacatcaaagaagttaacgaagctaactacccgcactactgcgatacaacccagtcttacggctgcccggcgggccaggcggcgtactacggtcgcggcccgatccagctttcttggaatttcaactataaagctgctggtgatgctctgggtattaacctgctggctaacccgtatctggttgaacaagatccggcagttgcttggaaaaccggtctgtggtattggaactctcagaacggtccgggtaccatgaccccgcacaacgctattgttaacaacgctggcttcggtgaaaccattcgttctattaacggtgctctggaatgtaacggtggtaacccggctcaggttcagtctcgtattaacaaattcactcagttcacccagatcctgggtaccaccaccggtccgaacctgtcttgcctcgag

SEQ ID NO: 19 - DNA, 27 bp, synthetic construct
  source 1..27: mol_type = other DNA
tctggatccggtgatttcgttaaaccg

SEQ ID NO: 20 - DNA, 29 bp, synthetic construct
  source 1..29: mol_type = other DNA
acactcgagccacgcatcccacagctgac

SEQ ID NO: 21 - DNA, 26 bp, synthetic construct
  source 1..26: mol_type = other DNA
tctggatccggtgatctggttaaacc

SEQ ID NO: 22 - DNA, 30 bp, synthetic construct
  source 1..30: mol_type = other DNA
acactcgagccaaacatcccacagctgatc

SEQ ID NO: 23 - DNA, 26 bp, synthetic construct
  source 1..26: mol_type = other DNA
tctggatcccacggttacgttgcttc

SEQ ID NO: 24 - DNA, 26 bp, synthetic construct
  source 1..26: mol_type = other DNA
acactcgaggatgttaacgacgatcg

SEQ ID NO: 25 - DNA, 29 bp, synthetic construct
  source 1..29: mol_type = other DNA
tctggatccggcccgaacgcgaacccgat

SEQ ID NO: 26 - DNA, 30 bp, synthetic construct
  source 1..30: mol_type = other DNA
acactcgagggtcggaacagacgcttcaga

SEQ ID NO: 27 - DNA, 27 bp, synthetic construct
  source 1..27: mol_type = other DNA
tctggatccggtccgaccaccccggtt

SEQ ID NO: 28 - DNA, 27 bp, synthetic construct
  source 1..27: mol_type = other DNA
acactcgaggataacctgaccgttgat

SEQ ID NO: 29 - DNA, 28 bp, synthetic construct
  source 1..28: mol_type = other DNA
tctggatccgccaacaacctgggttcta

SEQ ID NO: 30 - DNA, 31 bp, synthetic construct
  source 1..31: mol_type = other DNA
acactcgagtttctgcaggctcagaccatcg

SEQ ID NO: 31 - DNA, 30 bp, synthetic construct
  source 1..30: mol_type = other DNA
tctggatccgcgaccagcgcgaccgcgacc

SEQ ID NO: 32 - DNA, 31 bp, synthetic construct
  source 1..31: mol_type = other DNA
acactcgagagaggtaccatcacaagaacca

SEQ ID NO: 33 - DNA, 28 bp, synthetic construct
  source 1..28: mol_type = other DNA
tctggatccgccacctgcgcgaccgcgt

SEQ ID NO: 34 - DNA, 26 bp, synthetic construct
  source 1..26: mol_type = other DNA
acactcgaggcaagacaggttcggac

(19) United States
(12) Patent Application Publication
(10) Pub. No.: US 2025/0290162 A1
(43) Pub. Date: Sep. 18, 2025

(54) CHITIN-BINDING PROTEIN FOR DETECTING FUNGI AND APPLICATION THEREOF, AND AFFINITY MOLECULE DETECTION METHOD OF FUNGI

(71) Applicants: Chongqing Medical University, Chongqing (CN); Western (Chongqing) Collaborative Innovation Center for Intelligent Diagnostics and Digital Medicine, Chongqing (CN)

(72) Inventors: Deqiang Wang, Chongqing (CN); Shimei Shen, Chongqing (CN); Wen Wang, Chongqing (CN); Jie Wei, Chongqing (CN); Shaocheng Zhang, Chongqing (CN); Yuanyan Ma, Chongqing (CN); Xiaoli Wu, Chongqing (CN); Junye Liu, Chongqing (CN); Hua Zhou, Chongqing (CN); Yangli Zhang, Chongqing (CN); Shilei Wang, Chongqing (CN); Xuefei Cai, Chongqing (CN); Ailong Huang, Chongqing (CN)

(21) Appl. No.: 18/752,723

(22) Filed: Jun. 24, 2024

(30) Foreign Application Priority Data
Mar. 18, 2024 (CN) 202410304974.2

(51) Int. Cl. C12Q 1/6895; C12N 9/10; C12Q 1/6806; C12Q 1/6851

(52) U.S. Cl. CPC C12Q 1/6895; C12N 9/1051; C12Q 1/6806; C12Q 1/6851; C12Y 204/01016

(57) ABSTRACT

A chitin-binding protein for detecting fungi and an application thereof, and an affinity molecule detection method of the fungi are provided, relating to the field of fungus detection technologies. The chitin-binding protein includes ChBD2, ChBD3, EfCBP-1, PfCBP-A, PfCBP-B, BcCBP-1, or ScCBP-1. The chitin-binding protein can be used to detect the fungi. The affinity molecule detection method of the fungi can quickly and accurately quantitatively detect Candida albicans by combining a chitin affinity protein with recombinase polymerase amplification-clustered regularly interspaced short palindromic repeats/CRISPR related protein 12a (RPA-CRISPR/Cas12a). The whole reaction can be completed within 120 minutes, and the detection sensitivity of Candida albicans reaches 3×10¹ colony-forming units per milliliter (CFU/mL).

### CROSS-REFERENCE TO RELATED APPLICATION

This application claims priority to Chinese patent application No. CN202410304974.2, filed to China National Intellectual Property Administration (CNIPA) on Mar. 18, 2024, which is herein incorporated by reference in its entirety.

### TECHNICAL FIELD

The disclosure relates to the field of fungus detection technologies, and more particularly to a chitin-binding protein for detecting fungi and an application thereof, and an affinity molecule detection method of the fungi.

### STATEMENT REGARDING SEQUENCE LISTING

The sequence listing associated with this application is provided in text format in lieu of a paper copy and is hereby incorporated by reference into the specification. The name of the XML file containing the sequence listing is 24016THXT-USP1-SL.xml. The XML file is 40,454 bytes; is created on Jun. 12, 2024; and is being submitted electronically via patent center.

### BACKGROUND

Up to now, breakthroughs including in antibiotic use, cancer treatment, transplant surgery and invasive treatment, as well as the prevalence of human immunodeficiency virus (HIV) and severe acute respiratory syndrome coronavirus 2 (SARS-CoV-2), have led to a significant increase in the number of immunocompromised individuals. Therefore, invasive fungal infections (IFIs) have changed from a rare disease to an important factor of prevalence and increased mortality. Candida spp., Aspergillus spp, Cryptococcus spp, Pneumocystis and Mucor are the main pathogens causing the IFIs, among which Candida albicans is one of the common pathogens in clinic. In the related art, there are mainly three kinds of drugs for treating the IFIs: azoles, echinocandins and polyenes, but all of them have the disadvantage of potential toxicity. The nonspecific symptoms of the IFIs and the lack of timely diagnosis technology often lead to missed diagnosis, misdiagnosis and treatment failure. In this situation, there is currently a lack of timely detection methods to evaluate the efficacy of patients' antifungal treatment in time, which increases the complexity of managing these infections.

Cell walls of fungi are composed of glycoprotein, dextran and chitin. Chitin is a polysaccharide synthesized by a broad family of chitin synthase (CHS) enzymes and covalently bound to the dextran to form a complex polysaccharide network, which forms a firm structure and clastic basis of fungal cell walls and forms an extracellular matrix to escape recognition and immune capture by phagocytes. In addition, chitin and CHS genes have not been found in mammals, plants and bacteria, and only NodC protein encoded by Rhizobium (α-Alphaprotcobacteria) has homology with the CHS. Therefore, the chitin and the CHS are considered as specific targets for fungal diagnosis and antifungal therapy. Traditional fungal detection methods, including fungal culture, morphological detection, antigen and antibody detection, are widely used in clinic, but the methods based on culture have some problems, such as long turnover period and low sensitivity. The sensitivity of blood culture for invasive candidiasis (IC) detection is about 50%-90%. In an autopsy-confirmed IC patient, the sensitivity of blood culture before death is 21%-71%. Invasive aspergillosis (IA) is rare in blood culture, and its sensitivity is only 1%-5%. The average time for Candida blood culture to be positive is 2-3 days, and the longest is 7-8 days. Morphological detection and antigen-antibody detection have invasive sampling, subjectivity, and low sensitivity and specificity. Molecular techniques such as polymerase chain reaction (PCR) and matrix-assisted laser desorption ionization/time-of-flight (MALDI-TOF) mass spectrometry provide another effective choice for identifying fungal pathogens. However, some shortcomings limit their clinical application: PCR detection requires high-purity nucleic acid samples. The commercial nucleic acid extraction kit adopts solid phase extraction column, which may lead to DNA loss due to low adsorption efficiency and insufficient elution. In addition, the efficiency of the kit in blocking interference from the human genome is low. Moreover, both the PCR and the MALDI-TOF mass spectrometry need expensive equipment and skilled technicians, which limits their applicability in the on-site testing or clinical environment. On the contrary, isothermal amplification techniques, including loop-mediated isothermal amplification (LAMP) and recombinase polymerase amplification (RPA), can amplify nucleic acids at constant temperature, which makes them very suitable for low-resource environment applications and on-site diagnosis. Compared with the LAMP, the RPA simplifies the design of detection primers, and the reduction of the number of primers reduces the risk of primer interaction or cross-reaction. The nucleic acid amplification can be completed by incubating at 37-42° C. for 10-20 minutes with simple thermostatic equipment. In view of these advantages, the RPA has been widely used in the detection of a variety of pathogens. In addition, the combination of RPA and clustered regularly interspaced short palindromic repeats (CRISPR) related protein system (Cas) enhances the sensitivity, specificity, programmability and visual detection of diagnostic technology. Under the guidance of CRISPR RNA (crRNA), Cas12a protein specifically identifies the TTTN site of the protospacer adjacent motif (PAM) of the target sequence and binds to the double-stranded DNA (dsDNA). Subsequently, the cis-cleavage activity and trans-cleavage activity of the Cas12a enzyme are activated to specifically cleave the target dsDNA and non-specifically cleave a single-stranded DNA (ssDNA) reporter probe in the system, so that the excited fluorescence signal can be observed by naked eyes under the irradiation of blue light or ultraviolet light, and the fluorescence intensity can also be quantified in real time by a fluorescence detector. However, the combination of RPA and CRISPR/Cas12a faces challenges in practical application, including the interference between RPA and CRISPR/Cas12a systems. In addition, the high amplification efficiency of RPA introduces the potential risk of aerosol pollution, especially in the opening operation procedure. At present, the detection methods only pay attention to the specific components of fungi, such as fungal proteins, polysaccharides or nucleic acids, and do not consider a fungal spore with complete structure and infectious activity, so it can not accurately reflect the existence of infectious fungi.

### SUMMARY

A purpose of the disclosure is to provide a chitin-binding protein for detecting fungi and its application. The combination of chitin-binding protein with rapid amplification and one-pot detection method can quickly and accurately detect the fungi.

In order to achieve the above purpose, the disclosure provides the following technical solutions.

In an aspect, the disclosure provides a chitin-binding protein for detecting fungi, which includes ChBD2, ChBD3, EfCBP-1, PfCBP-A, PfCBP-B, BcCBP-1, or ScCBP-1.

The amino acid sequence of ChBD2 is shown in SEQ ID NO: 1, and the nucleotide sequence encoding the amino acid of ChBD2 is shown in SEQ ID NO: 2.

The amino acid sequence of ChBD3 is shown in SEQ ID NO: 3, and the nucleotide sequence encoding the amino acid of ChBD3 is shown in SEQ ID NO: 4.

The amino acid sequence of EfCBP-1 is shown in SEQ ID NO: 5, and the nucleotide sequence encoding the amino acid of EfCBP-1 is shown in SEQ ID NO: 6.

The amino acid sequence of PfCBP-A is shown in SEQ ID NO: 7, and the nucleotide sequence encoding the amino acid of PfCBP-A is shown in SEQ ID NO: 8.

The amino acid sequence of PfCBP-B is shown in SEQ ID NO: 9, and the nucleotide sequence encoding the amino acid of PfCBP-B is shown in SEQ ID NO: 10.

The amino acid sequence of BcCBP-1 is shown in SEQ ID NO: 11, and the nucleotide sequence encoding BcCBP-1 amino acid is shown in SEQ ID NO: 12.

The amino acid sequence of ScCBP-1 is shown in SEQ ID NO: 13, and the nucleotide sequence encoding ScCBP-1 amino acid is shown in SEQ ID NO: 14.

In another aspect, the disclosure also provides an application of the chitin-binding protein in detecting the fungi.

In still another aspect, the disclosure also provides an affinity molecule method of the fungi, which includes the following steps:
step (1), performing mixed incubation on the chitin-binding protein and carboxyl magnetic beads (MB) to obtain a magnetic bead-chitin-binding protein (MB-CBP) complex;
step (2), performing mixed incubation on the MB-CBP complex and a detection sample to obtain a MB-CBP-fungi complex (i.e., mixture);
step (3), extracting a genomic DNA of the MB-CBP-fungi complex;
step (4), performing rapid amplification and one-pot detection on the genomic DNA.

In an embodiment, in the step (1), a temperature of the mixed incubation is in a range of 20-30 Celsius degrees (° C.), and a time of the mixed incubation is in a range of 0.5-1.0 hours (h). In the step (1), a volume-mass ratio of the chitin-binding protein and the magnetic beads is in a range of 0.002-0.04:1.

In an embodiment, in the step (2), the detection sample is blood or bronchoalveolar lavage.

In an embodiment, in the step (2), a mixed volume ratio of the MB-CBP complex and the detection sample is in a range of 0.04-0.06:0.5-1.5. A temperature of the mixed incubation in the step (2) is in a range of 20-30° C. and a time of the mixed incubation is in a range of 20-35 minutes (min).

In an embodiment, in the step (3), a method for the extracting the genomic DNA of the mixture is a silica hydroxyl magnetic bead method.

In an embodiment, in the step (4), a method for the rapid amplification and one-pot detection of the genomic DNA is recombinase polymerase amplification-clustered regularly interspaced short palindromic repeats/CRISPR related protein 12a (RPA-CRISPR/Cas12a).

Specific steps of the RPA-CRISPR/CAS12a are as follows:
step a, performing RPA amplification on the genomic DNA and then collecting the amplified RPA system;
step b, performing mixed incubation on the amplified RPA system and a CRISPR/Cas12a system for 5-20 min to obtain a product, and then detecting the product to judge whether there are the fungi based on intensity of a fluorescence signal.

In an embodiment, the CRISPR/Cas12a system includes the following components: a 10×NE buffer2.1 (purchase from Sangon biotch, Shanghai, China), a CRISPR RNA (crRNA), a Cas12a protein, and an oligonucleotide probe.

An addition amount of the 10×NE buffer2.1 is in a range of 0.1-0.6 microliters (μL).
an addition amount of the crRNA is in a range of 1-4 μL, and an initial concentration of the crRNA is 1-3 micromoles per liter (μmol/L).

An addition amount of the Cas12a protein is in a range of 0.10-1.0 μL, and an initial concentration of the Cas12a protein is in a range of 4-5 μmol/L.

An addition amount of the oligonucleotide probe is in a range of 0.10-1.5 μL, and an initial concentration of the oligonucleotide probe is in a range of 1-15 μmol/L.

The nucleotide sequence of the crRNA is shown in SEQ ID NO: 15.

The nucleotide sequence of the Cas12a protein is shown in SEQ ID NO: 16.

The nucleotide sequence of the oligonucleotide probe is 5′-FAM-TTATTATT-BHQ1-3′.

In an embodiment, a temperature of the mixed incubation in the step b is in a range of 36-44° C.

The disclosure has the beneficial effects as follows.

The disclosure designs a novel affinity molecule diagnosis method, which combines chitin affinity magnetic separation method with RPA-CRISPR/Cas12a to rapidly and accurately quantitatively detect Candida albicans. The chitin-binding protein (CBP) is prepared and its binding affinity with chitin is evaluated. Then, the CBP-coupled magnetic beads are used to efficiently capture the fungi from whole blood or bronchoalveolar lavage fluid samples. Furthermore, the genomic DNA of the fungi is extracted and purified by silica hydroxyl magnetic beads, and the nucleic acid is rapidly amplified and detected by the RPA-CRISPR/Cas12a system. The whole reaction can be completed within 120 min, and the detection sensitivity of Candida albicans reaches 3×10¹ colony-forming units per milliliter (CFU/mL). The diagnosis platform is expected to provide low-cost, high-benefit, rapid, accurate and point-of-care testing (POCT) for invasive fungal infections (IFIs).

### BRIEF DESCRIPTION OF DRAWINGS

FIGS. 1A-1E illustrate a cell wall structure, a special component, a chitin-binding protein (CBP) structure, and a process of an affinity molecule detection method of fungi. Specifically, FIG. 1A illustrates the cell wall structure of the fungi, FIG. 1B illustrates the special component of fungi, FIG. 1C illustrates the chitin-binding protein structure, and FIGS. 1D-1E illustrate the process of the affinity molecule detection method of the fungi.

FIG. 2 illustrates a purified electropherogram of a SgCBP-1 protein.

FIGS. 3A-3B illustrate a purified electropherogram of chitin-binding proteins and affinity results of the chitin-binding proteins. Specifically, FIG. 3A illustrates the purified electropherogram of the chitin-binding proteins and FIG. 3B illustrates the affinity results of the chitin-binding proteins.

FIGS. 4A-4B illustrate coupling results evaluated by 15% sodium dodecyl sulfate polyacrylamide gel electrophoresis (SDS-PAGE) and Coomassie brilliant blue staining in an embodiment 3. Specifically, FIG. 4A illustrates electrophoresis results of magnetic bead-chitin-binding protein (MB-CBP) complexes after seven chitin-binding proteins are coupled with magnetic beads, and FIG. 4B illustrates electrophoresis results of supernatants corresponding to the coupling of the seven chitin-binding proteins.

FIGS. 5A-5H illustrate detection results of the MB-CBP complexes. Specifically, FIG. 5A illustrates change of protein concentration before and after coupling a PfCBP-A protein with carboxyl magnetic beads, FIG. 5B illustrates particle sizes before and after coupling the PfCBP-A protein with the carboxyl magnetic beads by a particle size analyzer, FIG. 5C illustrates electrophoresis results of MB-PfCBP-A complexes and corresponding supernatants after coupling 1 milligram (mg) of the PfCBP-A protein with different amounts (10 micrograms abbreviated as 10 μg, 20 μg and 30 μg) and the amount of protein required to saturate 1 mg of the carboxyl magnetic beads is detected, FIG. 5D illustrates electrophoresis results of the MB-CBP complexes after seven chitin-binding proteins are coupled with the magnetic beads in unsaturated state, FIG. 5E illustrates absorbance values at 260 nanometers (nm) of DNA supernatants of in an embodiment 4, FIG. 5F illustrates detection results of purity of genomic DNA of Candida albicans captured by the MB-CBP complexes, FIG. 5G illustrates detection results of content of the genomic DNA of Candida albicans captured by the MB-CBP complexes, and FIG. 5H illustrates absorbance values of the genomic DNA in an embodiment 5 at 260 nm.

FIGS. 6A-6G illustrate clinical verification results of the affinity molecule detection method. Specifically, FIG. 6A illustrates stability and specificity detection results, FIG. 6B illustrates fluorescence signal results observed by naked eyes, FIG. 6C illustrates real-time fluorescence monitoring results of blood samples, FIG. 6D illustrates real-time fluorescence monitoring results of bronchoalveolar lavage fluid (BALF) samples, FIG. 6E illustrates detection results of positive samples, FIG. 6F illustrates detection results of negative samples, and FIG. 6G illustrates fungal fluorescence staining microscope detection results.

FIGS. 7A-7B illustrate results of quantitative polymerase chain reaction (qPCR) detection. Specifically, FIG. 7A illustrates results of amplification refractory mutation system-qPCR (AMS-qPCR) detection on simulated blood and BALF samples, and FIG. 7B illustrates results of qPCR detection on simulated clinical samples by directly extracting nucleic acids without being captured by affinity magnetic beads.

### DETAILED DESCRIPTION OF EMBODIMENTS

The disclosure provides a chitin-binding protein for detecting fungi, which includes ChBD2, ChBD3, EfCBP-1, PfCBP-A, PfCBP-B, BcCBP-1 or ScCBP-1.

The amino acid sequence of ChBD2 is shown in SEQ ID NO: 1, and the nucleotide sequence encoding the amino acid of ChBD2 is shown in SEQ ID NO: 2.

The amino acid sequence of ChBD3 is shown in SEQ ID NO: 3, and the nucleotide sequence encoding the amino acid of ChBD3 is shown in SEQ ID NO: 4.

The amino acid sequence of EfCBP-1 is shown in SEQ ID NO: 5, and the nucleotide sequence encoding the amino acid of EfCBP-1 is shown in SEQ ID NO: 6;

The amino acid sequence of PfCBP-A is shown in SEQ ID NO: 7, and the nucleotide sequence encoding the amino acid of PfCBP-A is shown in SEQ ID NO: 8;

The amino acid sequence of PfCBP-B is shown in SEQ ID NO: 9, and the nucleotide sequence encoding the amino acid of PfCBP-B is shown in SEQ ID NO: 10;

The amino acid sequence of BcCBP-1 is shown in SEQ ID NO: 11, and the nucleotide sequence encoding BcCBP-1 amino acid is shown in SEQ ID NO: 12;

The amino acid sequence of ScCBP-1 is shown in SEQ ID NO: 13, and the nucleotide sequence encoding ScCBP-1 amino acid is shown in SEQ ID NO: 14.

The disclosure also provides an application of the chitin-binding protein in detecting the fungi.

In the disclosure, in view of the cell wall structure of the fungi and the special component of the fungi, the unique component chitin is selected as a target for capturing fungi. Chitin-binding protein (CBP) belongs to a family of carbohydrate-binding modules. Its crystal structure shows that hydrophilic residues and aromatic residues on its surface form a groove with strong negative charges, which interacts with the chitin.

The disclosure also provides an affinity molecule detection method of the fungi, which includes the following steps:
step (1), performing mixed incubation on the chitin-binding protein and magnetic beads to obtain a MB-CBP complex;
step (2), performing mixed incubation on the MB-CBP complex and a detection sample to obtain a mixture;
step (3), extracting a genomic DNA of the mixture; and
step (4), performing rapid amplification and one-pot detection on the genomic DNA.

In the disclosure, in the step (1), a temperature of the mixed incubation is in a range of 20-30 Celsius degrees (° C.). In a specific embodiment, the temperature of the mixed incubation in the step (1) is 25° C. A time of the mixed incubation in the step (1) is in a range of 0.5-2.0 hours (h). In a specific embodiment, the time of the mixed incubation in the step (1) is 1.25 h.

A volume-to-mass ratio of the chitin-binding protein and the magnetic beads is in a range of 0.002-0.04:1. In a specific embodiment, the volume-to-mass ratio of the chitin-binding protein and the magnetic beads is 0.021:1.

In the disclosure, in the step (2), the detection sample is at least one of blood and bronchoalveolar lavage fluid.

In the disclosure, in the step (2), a mixed volume ratio of the MB-CBP complex and the detection sample is in a range of 0.04-0.06:0.5-1.5. In a specific embodiment, the mixed volume ratio of the MB-CBP complex and the detection sample is 0.05:1.0.

A temperature of the mixed incubation in the step (2) is in a range of 20-30° C. and a time of the mixed incubation in the step (2) is in a range of 20-35 minutes (min). In a specific embodiment, the temperature of the mixed incubation in the step (2) is 25° C., and the time of the mixed incubation in the step (2) is 27.5 min.

In the disclosure, in the step (3), a method for the extracting the genomic DNA of the mixture is a silica hydroxyl magnetic bead method.

In the disclosure, in the step (4), a method for the rapid amplification and one-pot detection of the genomic DNA is recombinase polymerase amplification-clustered regularly interspaced short palindromic repeats/CRISPR related protein 12a (RPA-CRISPR/Cas12a).

Specific steps of the RPA-CRISPR/CAS12a are as follows:
step a, performing RPA amplification on the genomic DNA and then collecting an amplified RPA system; and
step b, performing mixed incubation on the amplified RPA system and a CRISPR/Cas12a system for 5-20 min to obtain a product, and then detecting the product to judge whether there are the fungi based on intensity of a fluorescence signal.

In the disclosure, in a specific embodiment, a time of the mixed incubation in the step b is 12.5 min.

In the disclosure, the CRISPR/Cas12a system includes the following components: a 10×NE buffer2.1 (purchase from Sangon biotch, Shanghai, China), a CRISPR RNA (crRNA), a Cas12a protein, and an oligonucleotide probe.

An addition amount of the 10×NE buffer2.1 is in a range of 0.1-0.6 microliters (μL). In a specific embodiment, the addition amount of the 10×NE buffer2.1 is 0.35 μL.

An addition amount of the crRNA is in a range of 1-4 μL. In a specific embodiment, the addition amount of the crRNA is 2.5 μL. An initial concentration of the crRNA is 1-3 in a range of micromoles per liter (μmol/L). In a specific embodiment, the initial concentration of the crRNA is 1.5 μmol/L.

An addition amount of the Cas12a protein is in a range of 0.10-1.0 μL. In a specific embodiment, the addition amount of the Cas12a protein is 0.55 μL. An initial concentration of the Cas12a protein is in a range of 4-5 μmol/L. In a specific embodiment, the initial concentration of the Cas12a protein is 4.5 μmol/L.

An addition amount of the oligonucleotide probe is in a range of 0.10-1.5 μL. In a specific embodiment, the addition amount of the oligonucleotide probe is 0.8 μL. An initial concentration of the oligonucleotide probe is in a range of 1-15 μmol/L. In a specific embodiment, the initial concentration of the oligonucleotide probe is 8 μmol/L.

The nucleotide sequence of the crRNA is shown in SEQ ID NO: 15: UAAUUUCUACUAAGUGUAGAUCAUAGAUUAUAAAUGGCAUGG.

The nucleotide sequence coding the Cas12a protein is shown in SEQ ID NO: 16:

GCAGCAAGTAAACTGGAAAAATTTACCAATTGTTATAGTCTGAGCAAAACACTGC
GTTTTAAAGCAATTCCGGTTGGTAAAACCCAGGAAAATATTGATAATAAACGCCT
GCTGGTTGAAGATGAAAAACGTGCAGAAGATTATAAAGGTGTGAAAAAGCTGCT
GGATCGTTATTATCTGAGTTTTATTAACGATGTGCTGCATAGCATTAAACTGAAA
AACCTGAATAACTATATCAGCCTGTTTCGTAAAAAGACCCGTACCGAAAAAGAA
AATAAAGAACTGGAAAACCTGGAAATTAACCTGCGCAAAGAAATCGCCAAAGCA
TTTAAAGGCGCCGCAGGCTATAAAAGCCTGTTTAAAAAGGATATTATCGAAACAA
TCCTGCCGGAAGCAGCAGATGATAAAGATGAAATCGCACTGGTGAATAGCTTTA
ATGGCTTTACCACAGCATTTACCGGTTTTTTTGATAATCGTGAAAACATGTTTAGT
GAAGAAGCAAAAAGCACCAGCATTGCATTTCGCTGTATTAACGAAAATCTGACCC
GCTATATTAGCAATATGGATATTTTTGAAAAAGTTGATGCAATCTTTGATAAACA
TGAAGTTCAGGAAATCAAAGAAAAAATTCTGAATAGCGATTATGATGTTGAAGA
TTTTTTTGAAGGTGAATTTTTTAATTTTGTTCTGACCCAGGAAGGTATTGATGTTT
ATAATGCAATTATTGGTGGTTTTGTTACCGAAAGCGGTGAAAAAATTAAAGGTCT
GAATGAATATATTAATCTGTATAATGCAAAAACCAAACAGGCACTGCCGAAATTT
AAACCGCTGTATAAACAGGTTCTGAGCGATCGTGAAAGCCTGAGCTTTTATGGTG
AAGGTTATACCAGCGATGAAGAAGTTCTGGAAGTTTTTCGTAATACCCTGAATAA
AAATAGCGAAATTTTTAGCAGCATTAAAAAACTGGAAAAACTGTTTAAAAATTTT
GATGAATATAGCAGCGCAGGTATTTTTGTTAAAAATGGTCCGGCAATTAGCACCA
TTAGCAAAGATATTTTTGGTGAATGGAATCTGATTCGTGATAAATGGAATGCAGA
ATATGATGATATTCATCTGAAAAAAAAAGCAGTTGTTACCGAAAAATATGAAGAT
GATCGTCGTAAAAGTTTTAAAAAAATTGGTAGCTTTAGCCTGGAACAGCTGCAGG
AATATGCAGATGCAGATCTGAGCGTTGTTGAAAAACTGAAAGAAATTATTATTCA
GAAAGTTGATGAAATTTATAAAGTTTATGGTAGCAGCGAAAAACTGTTTGATGCA
GATTTTGTTCTGGAAAAAAGCCTGAAAAAAAATGATGCAGTTGTTGCAATTATGA
AAGATCTGCTGGATAGCGTTAAAAGTTTTGAAAATTATATTAAAGCATTTTTTGGT
GAAGGTAAAGAAACCAATCGTGATGAAAGTTTTTATGGTGATTTTGTTCTGGCAT
ACGATATTCTGCTGAAAGTTGATCATATTTATGATGCAATTCGTAATTATGTTACC
CAGAAACCGTATAGCAAAGATAAATTTAAACTGTATTTTCAGAATCCGCAGTTTA
TGGGTGGTTGGGATAAAGATAAAGAAACCGATTATCGTGCAACCATTCTGCGTTA
TGGTAGCAAATATTATCTGGCAATTATGGATAAAAAATATGCAAAATGTCTGCAG
AAAATTGATAAAGATGATGTTAATGGTAATTATGAAAAAATTAATTATAAACTGC
TGCCGGGTCCGAATAAAATGCTGCCGAAAGTTTTTTTTAGCAAAAAATGGATGGC
ATATTATAATCCGAGCGAAGATATTCAGAAAATTTATAAAAATGGTACATTTAAA
AAAGGTGATATGTTTAATCTGAATGATTGTCATAAACTGATTGATTTTTTTAAAGA
TAGCATTAGCCGTTATCCGAAATGGAGCAATGCATACGATTTTAATTTTAGCGAA
ACCGAAAAATATAAAGATATTGCAGGTTTTTATCGTGAAGTTGAAGAACAGGGTT
ATAAAGTTAGCTTTGAAAGCGCAAGCAAAAAAGAAGTTGATAAACTGGTTGAAG
AAGGTAAACTGTATATGTTTCAGATTTATAATAAAGATTTTAGCGATAAAAGCCA
CGGTACACCGAATCTGCATACCATGTATTTTAAACTGCTGTTTGATGAAAATAAT
CATGGTCAGATTCGTCTGAGCGGTGGTGCAGAACTGTTTATGCGTCGTGCAAGCC
TGAAAAAAGAAGAACTGGTTGTTCATCCGGCAAATAGCCCGATTGCAAATAAAA
ATCCGGATAATCCGAAAAAAACCACCACCCTGAGCTATGATGTTTATAAAGATAA
ACGTTTTAGCGAAGATCAGTATGAACTGCATATTCCGATTGCAATTAATAAATGT
CCGAAAAATATTTTTAAAATTAATACCGAAGTTCGTGTTCTGCTGAAACATGATG
ATAATCCGTATGTTATTGGTATTGATCGTGGTGAACGTAATCTGCTGTATATTGTT
GTTGTTGATGGTAAAGGTAATATTGTTGAACAGTATAGCCTGAATGAAATTATTA
ATAATTTTAATGGTATTCGTATTAAAACCGATTATCATAGCCTGCTGGATAAAAA
AGAAAAAGAACGTTTTGAAGCACGTCAGAATTGGACCAGCATTGAAAATATTAA
AGAACTGAAAGCAGGTTATATTAGCCAGGTTGTTCATAAAATTTGTGAACTGGTT
GAAAAATATGATGCAGTTATTGCACTGGAAGATCTGAATAGCGGTTTTAAAAATA
GCCGTGTTAAAGTTGAAAAACAGGTTTATCAGAAATTTGAAAAAATGCTGATTGA
TAAACTGAATTATATGGTTGATAAAAAAAGCAATCCGTGTGCAACCGGTGGTGCA
CTGAAAGGTTATCAGATTACCAATAAATTTGAAAGTTTTAAAAGCATGAGCACCC
AGAATGGTTTTATTTTTTATATTCCGGCATGGCTGACCAGCAAAATTGATCCGAGC
ACCGGTTTTGTTAATCTGCTGAAAACCAAATATACCAGCATTGCAGATAGCAAAA
AATTTATTAGCAGCTTTGATCGTATTATGTATGTTCCGGAAGAAGATCTGTTTGAA
TTTGCACTGGATTATAAAAATTTTAGCCGTACCGATGCAGATTATATTAAAAAAT
GGAAACTGTATAGCTATGGTAATCGTATTCGTATTTTTGCAGCAGCAAAAAAAAA
TAATGTTTTTGCATGGGAAGAAGTTTGTCTGACCAGCGCATATAAAGAACTGTTT
AATAAATATGGTATTAATTATCAGCAGGGTGATATTCGTGCACTGCTGTGTGAAC
AGAGCGATAAAGCATTTTATAGCAGCTTTATGGCACTGATGAGCCTGATGCTGCA
GATGCGTAATAGCATTACCGGTCGTACCGATGTTGATTTTCTGATTAGCCCGGTTA
AAAATAGCGATGGTATTTTTTATGATAGCCGTAATTATGAAGCACAGGAAAATGC
AATTCTGCCGAAAAATGCAGATGCAAATGGTGCATATAATATTGCACGTAAAGTT
CTGTGGGCAATTGGTCAGTTTAAAAAAGCAGAAGATGAAAAACTGGATAAAGTT
AAAATTGCAATTAGCAATAAAGAATGGCTGGAATATGCACAGACCAGCGTTAAA.

The nucleotide sequence of the oligonucleotide probe is 5′-FAM-TTATTATT-BHQ1-3′.

In the disclosure, a temperature of the mixed incubation in the step b is in a range of 36-44° C. In a specific embodiment, the temperature of the mixed incubation in the step b is 40° C.

In the CRISPR/Cas12a reaction process, it is necessary to fix a polytetrafluoroethylene capillary with an inner diameter of 1 millimeter (mm) inside an Eppendorf (EP) tube or an eight-tube strip cap to form a tube-in-tube structure, the RPA system is added to the bottom of the tube during the reaction, and the CRISPR/Cas12a reaction system is placed in a capillary connected with the tube cap. After RPA amplification reaction is completed by incubation at 42° C. for 20 min, the CRISPR/Cas12a system and the RPA system are mixed at the bottom of the tube by short-term centrifugation to activate cis-cleavage and trans-cleavage activities of the Cas12a-crRNA complex. The tube-in-tube structure reduces the mutual interference between the two reaction systems. The operation is convenient, and the reaction process does not need to be opened, thus avoiding the risk of aerosol pollution.

The technical solutions provided by the disclosure will be described in detail with embodiments below, but they cannot be understood as limiting the protection scope of the disclosure.

### Embodiment 1

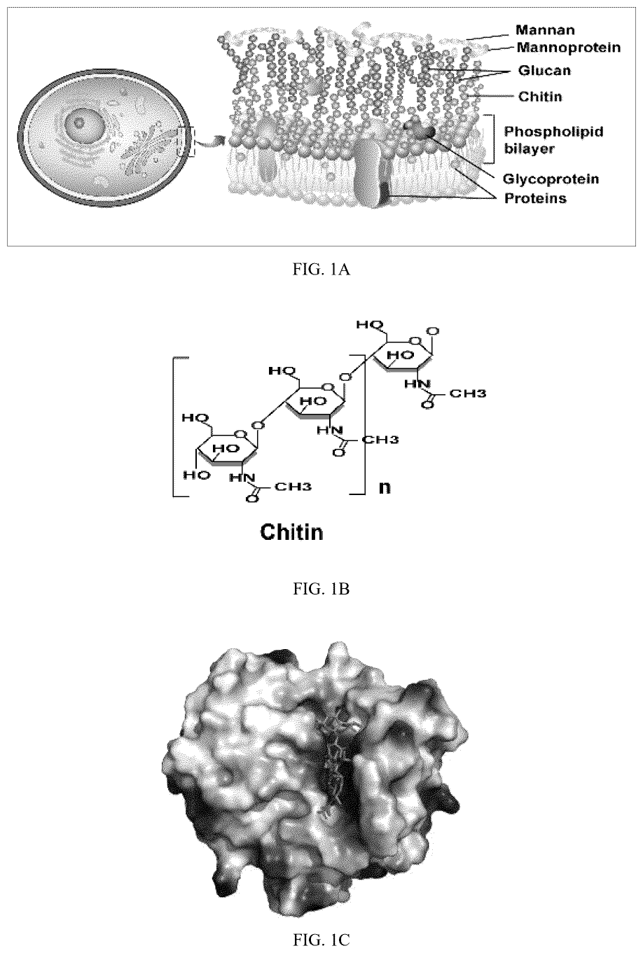

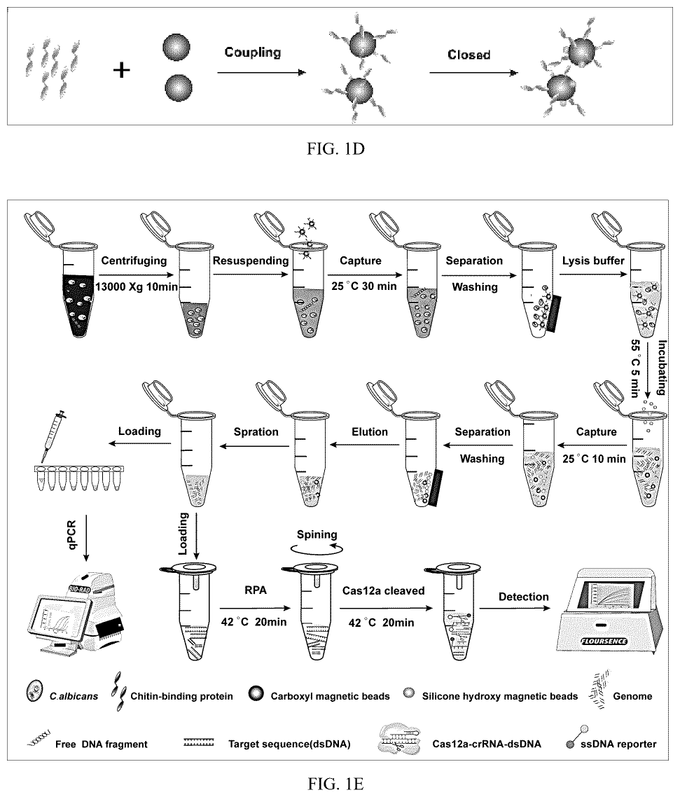

In the disclosure, in view of the cell wall structure of Candida albicans (FIG. 1A) and the special component chitin of fungi (FIG. 1B), the unique component chitin is selected as the target for capturing fungi. CBP (FIG. 1C) belongs to the family of carbohydrate-binding modules. Its crystal structure shows that hydrophilic residues and aromatic residues on its surface form a groove with strong negative charges, which interacts with chitin.

Specifically, an affinity molecule detection method of Candida albicans includes the following steps (as shown in FIGS. 1D-1E) as follows.

(1) 1 milligram (mg) of carboxyl magnetic beads is washed with 500 μL of 15 millimoles per liter (mmol/L) 2-Morpholinoethanesulphonic acid (MES) buffer solution for three times, then added 20 μL of N-Hydroxysuccinimide abbreviated as NHS (0.015 moles per liter abbreviated as mol/L, power of potential abbreviated as pH=5.5) and 20 μL of 1-Ethyl-3-(3-dimethylaminopropyl)carbodiimide hydrochloride abbreviated as EDC (10 milligrams per milliliter abbreviated as mg/mL) in turn, vortex oscillated for 30 seconds(s), and incubated at 25° C. in a four-dimensional mixing device for 30 min, so as to obtain a magnetic bead mixture. The magnetic bead mixture is separated with a magnetic rack to discard the supernatant, the separated magnetic bead mixture is washed twice with the MES, and resuspended to 10 mg/mL to obtain activated magnetic beads.

20 micrograms (μg) of chitin-binding protein and 1 mg of the activated magnetic beads (1 micrometer abbreviated as um particle size, 10 mg/mL) are incubated in the four-dimensional mixing device at 25° C. for 2.0 h to obtain a MB-CBP complex.

The esterification reaction is carried out with the carboxyl magnetic beads in a carboxyl magnetic bead blocking solution at 25° C. for 1 h to block residual carboxyl on the magnetic beads, and the MB-CBP complex is washed twice with 200 μL of 15 mmol/L phosphate-buffered saline with Tween® 20 (PBST) pH7.4 and resuspended to 10 mg/mL.

(2) 50 μL of the MB-CBP complex and 1 mL of detection sample are mixed and incubated at 25° C. for 30 min to obtain a mixture.

Specifically, the detection sample is taken and centrifuged at 12000 revolutions per minute (rpm) for 10 min, most of the supernatant and blood cells are gently discarded, 200 μL precipitate is remained, the MB-CBP complex is added and incubated in the four-dimensional mixing device at 25° C. for 30 min to obtain the mixture.

(3) The genomic DNA is extracted.

Specifically, the mixture is gently washed with 15 mmol/L phosphate-buffered saline (PBS) for 3 times, and the supernatant is discarded after the washed mixture is separated with the magnetic rack to obtain precipitate 1.

450 μL of recombinant cell fungal lysate (1 mol/L guanidine hydrochloride, 37.5 mL 75% ethanol, 1 mL Triton-X-100, 5 mmol/L Tris(hydroxymethyl)aminomethane hydrochloride (Tris-HCl), 5 mmol/L ethylenediaminetetraacetic acid (EDTA), 0.1 mg/mL protease K, pH 8.0) is added to the precipitate 1. After lysis at 55° C. for 5 min, 100 μL of 50 mg/mL silica hydroxyl magnetic beads and 2 μL of 10 mg/mL RNase A are added and incubated at 25° C. for 10 min. The supernatant is discarded after separation with the magnetic rack to obtain precipitate 2.

500 μL of washing buffer 1 (1 mol/L guanidine hydrochloride, 37.5 mL 75% ethanol, 10 mmol/L Tris-HCl, 10 mmol/L EDTA, pH 8.0) is added to the precipitate 2 and washed twice. 700 μL of washing buffer 2 (80% ethanol) is added and washed once. 50 μL diethyl pyrocarbonate (DEPC)-treated water is added into the washed precipitate 2 and incubated at 55° C. for 5 min to elute the genomic DNA, and separated and collected supernatant containing DNA with the magnetic rack to obtain the genomic DNA.

(4) The rapid amplification and one-pot detection are performed on the extracted genomic DNA.

Specifically, 1.25 μL of 280 mmol/L magnesium acetate initiator is added into the RPA system, and incubated at 42° C. for 20 min after mixing evenly to obtain the amplified RPA system.

In this situation, the amplification system is 25 μL of RPA enzyme mixed solution, 0.5 μL each of 30 μmol/L of forward and reverse primers (as shown in Table 2) with, 1 μL of fungal genomic DNA and 15 μL of 2×RPA buffer, which are made up to 28.5 μL with distilled water, evenly mixed and instantaneously centrifuged (6000 rpm, 5 s).

Preparation of the RPA enzyme mixed solution: protease is taken out from −80° C. and slowly thawed at 4° C., the concentration of protease is measured by bicinchoninic acid assay (BCA) method, and the RPA enzyme mixed solution is prepared according to 25 μL reaction system. The final concentration of recombinant enzyme is 30 nanograms per microliter (ng/μL), the final concentration of recombinant enzyme loading factor is 50 ng/μL, the final concentration of polymerase is 50 ng/μL, and the final concentration of single-stranded binding protein is 50 ng/μL.

Preparation of the CRISPR/Cas12a system: 0.5 μL of 10×NE buffer2.1, 3 μL of 2.5 μmol/L crRNA (the nucleotide sequence as shown in SEQ ID NO: 15), 0.75 μL of 5 μmol/L Cas12a protein (the nucleotide sequence as shown in SEQ ID NO: 16), 1 μL of 15 μmmol/L oligonucleotide probe (the nucleotide sequence as shown in SEQ ID NO: 16); and the system is added to the capillary of the reaction tube cap.

The amplified RPA system and the CRISPR/Cas12a system are mixed and incubated at 42° C. for 20 min, and then detected. The fluorescence intensity generated by the reaction system is monitored in real time in a fluorescence detector, and whether there is Candida albicans is judged according to the fluorescence signal.

### Embodiment 2 Identification and Screening of Chitin-Binding Protein Affinity

### Plasmid Construction

(1) Preparation of target fragments: the amino acid sequences of target proteins (ChBD2, ChBD3, EfCBP-1, PfCBP-A, PfCBP-B, BcCBP-1, ScCBP-1, ScCBP-2) are found on National Center for Biotechnology Information (NCBI), then converted into nucleotide sequences (the corresponding amino acid sequences of the respective target proteins are shown in Table 1), and codon optimization is carried out and corresponding primers are designed (primers are shown in Table 2). The purified target fragments are obtained by PCR amplification using the bacteria solutions of Thermococcus kodakarensis, Enterococcus faecalis, Morganella morganii, Bacillus cereus, and Streptomyces coelicolor as templates. The target fragments are amplified by PCR and the amplified products are obtained. The amplification system (20 μL) is 10 μL of 2×Taq DNA polymerase, 0.5 μL of each of 10 micromoles per liter (μM) forward and reverse primers, 1 μL of bacteria solution, and 8 μL of enzyme-free water. The amplification procedures are 95° C. for 2 min, 55° C. for 30 s, 72° C. for 2 min, with 34 cycles.

TABLE 1
Table of amino acid sequences and nucleotide sequences corresponding to
the respective target proteins

| Protein | Amino acid sequence | Nucleotide sequence |
|---|---|---|
| ChBD2 | SEQ ID NO: 1: | SEQ ID NO: 2: |
| | GDFVKPGSLSVKVTDW | GGTGATTTCGTTAAACCGGGCAGCCTGAGCG |
| | GNTEYDVTLNLGGTYD | TTAAAGTTACCGATTGGGGCAACACCGAATA |
| | WVVKVKLKDGSSVSSF | CGATGTTACCCTGAACCTGGGCGGCACCTAC |
| | WSANKAEEGGYVVFTP | GATTGGGTTGTTAAAGTTAAACTGAAAGATG |
| | VSWNRGPTATFGFIATG | GCAGCAGCGTTAGCAGCTTCTGGAGCGCGA |
| | SESVEAIYLYVDGQLW | ACAAAGCGGAAGAAGGCGGCTACGTTGTTT |
| | DAW | TCACCCCGGTTTCTTGGAACCGTGGTCCGAC |
| | | CGCGACCTTCGGTTTCATCGCGACCGGTTCT |
| | | GAAAGCGTTGAAGCGATCTACCTGTACGTTG |
| | | ATGGTCAGCTGTGGGATGCGTGG |
| ChBD3 | SEQ ID NO: 3: | SEQ ID NO: 4: |
| | GSGDLVKPDAFSVKIQD | GGATCCGGTGATCTGGTTAAACCGGATGCGT |
| | WGSTEYDVTLNLGGTY | TCAGCGTTAAAATCCAGGATTGGGGCAGCAC |
| | DWVVKVKLKDGSAVS | CGAATACGATGTTACCCTGAACCTGGGCGGC |
| | SVWSANKAEEGGYVV | ACCTACGATTGGGTTGTTAAAGTTAAACTGA |
| | FTPVSWNKGPTATFGFI | AAGATGGCAGCGCGGTTAGCAGCGTTTGGA |
| | ATGSEPVEAMYLYVND | GCGCGAACAAAGCGGAAGAAGGCGGCTACG |
| | QLWDVWLE | TTGTTTTCACCCCGGTTAGCTGGAACAAAGG |
| | | CCCGACCGCGACCTTCGGCTTCATCGCGACC |
| | | GGCAGCGAACCGGTTGAAGCGATGTACCTGT |
| | | ACGTTAACGATCAGCTGTGGGATGTTTGGCT |
| | | CGAG |
| EfCBP-1 | SEQ ID NO: 5: | SEQ ID NO: 6: |
| | HGYVASPGSRAFFGSSA | CACGGTTACGTTGCTTCTCCGGGTAGCCGTG |
| | GGNLNTNVGRAQWEP | CGTTCTTCGGCAGCTCTGCGGGCGGTAACCT |
| | QSIEAPKNTFITGKLAS | GAACACCAACGTTGGCCGTGCGCAGTGGGA |
| | AGVSGFEPLDEQTATR | ACCGCAGAGCATCGAAGCGCCGAAAAACAC |
| | WHKTNITTGPLDITWNL | CTTCATCACCGGTAAACTGGCGAGCGCGGGC |
| | TAQHRTASWDYYITKN | GTTAGCGGTTTCGAACCGCTGGATGAACAGA |
| | GWNPNQPLDIKNFDKI | CCGCGACCCGTTGGCACAAAACCAACATCAC |
| | ASIDGKQEVPNKVVKQ | CACCGGCCCGCTGGATATCACCTGGAACCTG |
| | TINIPTDRKGYHVIYAV | ACCGCGCAGCACCGTACCGCGAGCTGGGATT |
| | WGIGDTVNAFYQAIDV | ACTACATCACCAAAAACGGCTGGAACCCGA |
| | NIQ | ACCAGCCGCTGGATATCAAAAACTTCGATAA |
| | | AATCGCGAGCATCGATGGCAAACAGGAAGTT |
| | | CCGAACAAAGTTGTTAAACAGACCATCAACA |
| | | TCCCGACCGATCGTAAAGGCTACCACGTTAT |
| | | CTACGCGGTGTGGGGCATTGGTGACACCGTT |
| | | AACGCATTTTATCAGGCGATCGACGTTAACAT |
| | | CCAG |
| PfCBP-A | SEQ ID NO: 7: | SEQ ID NO: 8: |
| | GSLSNQSGYPAFRGLM | GGATCCCTGAGCAACCAGAGCGGCTACCCG |
| | SWSINWDAKNNFEFSN | GCGTTCCGTGGCCTGATGAGCTGGTCCATCA |
| | NYRTYFDGLSLQKGPN | ACTGGGATGCGAAAAACAACTTCGAATTCAG |
| | ANPIPEHFFAPYIDMSLS | CAACAACTACCGTACCTACTTCGATGGTCTG |
| | VHKPLVEYAKLTGTKY | AGCCTGCAGAAAGGCCCGAACGCGAACCCG |
| | FTLAFILYSSVYNGPAW | ATCCCGGAACACTTCTTCGCGCCGTACATCG |
| | AGSIPLEKFVDEVRELR | ATATGTCTCTGTCTGTTCACAAACCGCTGGTT |
| | EIGGEVIIAFGGAVGPYL | GAATACGCTAAACTGACCGGTACCAAATACT |
| | CQQASTPEQLAEWYIK | TCACCCTGGCGTTCATCCTGTACTCCAGCGTT |
| | VIDTYNATYLDFDIAAG | TACAACGGCCCGGCGTGGGCGGGTAGCATCC |
| | IDADKLADALLIVQRER | CGCTGGAAAAATTCGTTGACGAAGTGCGTGA |
| | PWVKFSFTLPSDPGIGL | ACTGCGTGAAATCGGTGGTGAAGTGATCATC |
| | AGGYGIIETMAKKGVR | GCGTTCGGCGGCGCGGTTGGTCCGTACCTGT |
| | VDRVNPMTMDYYWTP | GCCAGCAGGCGAGCACCCCGGAACAGCTGG |
| | SNAENAIKVAENVFRQ | CGGAATGGTACATCAAAGTTATCGACACCTA |
| | LKQIYPEKSDEEIWKMI | CAACGCTACCTACCTGGATTTTGATATTGCGG |
| | GLTPMIGVNDDKSVFTL | CGGGTATCGATGCTGACAAACTGGCGGATGC |
| | EDAQQLVDWAIQHKIG | TCTGCTGATTGTTCAGCGTGAACGTCCGTGG |
| | SLAFWSVDRDHPGPTG | GTTAAATTCTCTTTCACCCTGCCGAGCGATCC |
| | EVSPLHRGTNDPDWAF | GGGCATCGGCCTGGCAGGTGGTTACGGTATC |
| | SHVFVKFMEAFGYTFS | ATCGAAACCATGGCGAAAAAAGGCGTTCGT |
| | AQTSEASVPTLE | GTTGATCGTGTTAACCCGATGACCATGGACTA |
| | | CTATTGGACCCCGTCTAACGCTGAAAACGCG |
| | | ATTAAAGTTGCGGAAAACGTTTTCCGTCAGC |
| | | TGAAACAGATCTACCCGGAAAAATCTGATGA |
| | | AGAAATCTGGAAAATGATCGGTCTGACCCCG |
| | | ATGATCGGTGTTAACGATGACAAATCTGTGTT |
| | | CACCCTGGAAGATGCTCAGCAGCTGGTTGAT |
| | | TGGGCGATCCAGCACAAAATTGGCTCTCTGG |
| | | CGTTCTGGAGCGTTGATCGTGATCACCCAGG |
| | | CCCGACCGGTGAAGTTTCTCCGCTGCACCGT |
| | | GGCACCAACGATCCGGATTGGGCTTTCAGCC |
| | | ACGTTTTCGTTAAATTCATGGAAGCATTCGGC |
| | | TACACCTTCAGCGCGCAGACCTCTGAAGCGT |
| | | CTGTTCCGACCCTCGAG |
| PfCBP-B | SEQ ID NO: 9: | SEQ ID NO: 10: |
| | GPTTPVPVSGSLEVKVN | GATGTTACCCTGAACCTGGATGGTCAGTACG |
| | DWGSGAEYDVTLNLD | ATTGGACCGTTAAAGTTAAACTGGCGCCGGG |
| | GQYDWTVKVKLAPGA | TGCGACCGTTGGTAGCTTCTGGTCTGCGAAC |
| | TVGSFWSANKQEGNG | AAACAGGAAGGTAACGGTTACGTTATCTTCA |
| | YVIFTPVSWNKGPTATF | CCCCGGTTTCTTGGAACAAAGGTCCGACCGC |
| | GFIVNGPQGDKVEEITL | GACCTTCGGTTTCATCGTTAACGGTCCGCAG |
| | EINGQVIL | GGTGATAAAGTTGAAGAAATCACCCTGGAAA |
| | | TCAACGGTCAGGTTATC |
| BcCBP-1 | SEQ ID NO: 11: | SEQ ID NO: 12: |
| | ANNLGSKLLVGYWHNF | GCCAACAACCTGGGTTCTAAACTGCTGGTTG |
| | DNGTGIIKLKDVSPKW | GTTACTGGCACAACTTCGATAACGGTACCGG |
| | DVINVSFGETGGDRSTV | TATCATCAAACTGAAAGATGTTAGCCCGAAA |
| | EFSPVYGTDADFKSDIS | TGGGATGTTATCAACGTTAGCTTCGGTGAAA |
| | YLKSKGKKVVLSIGGQ | CCGGTGGTGATCGTTCTACCGTTGAATTCAG |
| | NGVVLLPDNAAKDRFI | CCCGGTTTACGGTACCGATGCGGATTTCAAAT |
| | NSIQSLIDKYGFDGIDID | CCGATATCAGCTACCTGAAAAGCAAAGGTAA |
| | LESGIYLNGNDTNFKNP | AAAAGTTGTTCTGTCTATCGGCGGTCAGAAC |
| | TTPQIVNLISAIRTISDH | GGCGTTGTTCTGCTGCCGGATAACGCGGCGA |
| | YGPDFLLSMAPETAYV | AAGATCGTTTCATCAACAGCATCCAGTCTCT |
| | QGGYSAYGSIWGAYLPI | GATCGATAAATACGGTTTCGATGGTATCGATA |
| | IYGVKDKLTYIHVQHY | TCGATCTGGAAAGCGGTATCTACCTGAACGG |
| | NAGSGIGMDGNNYNQ | TAACGATACCAACTTCAAAAACCCGACCACC |
| | GTADYEVAMADMLLH | CCGCAGATCGTTAACCTGATCAGCGCAATCC |
| | GFPVGGNANNIFPALRS | GTACCATCTCTGATCACTACGGCCCGGATTTC |
| | DQVMIGLPAAPAAAPS | CTGCTGTCTATGGCGCCGGAAACCGCGTACG |
| | GGYISPTEMKKALNYII | TTCAGGGTGGTTACAGCGCGTACGGTTCTAT |
| | KGVPFGGKYKLSNQSG | CTGGGGTGCGTACCTGCCGATCATCTACGGT |
| | YPAFRGLMSWSINWDA | GTTAAAGATAAACTGACCTACATCCACGTTC |
| | KNNFEFSNNYRTYFDG | AGCACTACAACGCTGGTAGCGGTATCGGCAT |
| | LSLQK | GGATGGTAACAACTACAACCAGGGTACCGCG |
| | | GATTACGAAGTTGCGATGGCGGATATGCTGCT |
| | | GCACGGCTTCCCGGTTGGCGGTAACGCGAAC |
| | | AACATCTTCCCGGCGCTGCGTAGCGATCAGG |
| | | TTATGATCGGTCTGCCGGCGGCGCCGGCTGC |
| | | GGCTCCGAGCGGTGGCTACATCAGCCCGACC |
| | | GAAATGAAAAAAGCGCTGAACTACATCATCA |
| | | AAGGCGTTCCGTTCGGTGGCAAATACAAACT |
| | | GAGCAACCAGAGCGGCTACCCGGCGTTCCG |
| | | TGGCCTGATGAGCTGGTCCATCAACTGGGAT |
| | | GCGAAAAACAACTTCGAATTCAGCAACAACT |
| | | ACCGTACCTACTTCGATGGTCTGAGCCTGCA |
| | | GAAA |
| ScCBP-1 | SEQ ID NO: 13: | SEQ ID NO: 14: |
| | ATSATATFAKTSDWGTG | GCGACCAGCGCGACCGCGACCTTCGCGAAA |
| | FGGSWTVKNTGTTSLS | ACCAGCGATTGGGGCACCGGCTTCGGCGGC |
| | SWTVEWDFPTGTKVTS | AGCTGGACCGTTAAAAACACCGGCACCACC |
| | AWDATVTNSGDHWTA | AGCCTGAGCAGCTGGACCGTTGAATGGGATT |
| | KNVGWNGTLAPGASV | TCCCGACCGGCACCAAAGTTACCAGCGCGTG |
| | SFGFNGSGPGSPSNCKL | GGATGCGACCGTTACCAACAGCGGCGATCAC |
| | NGGSCDGTS | TGGACCGCGAAAAACGTTGGCTGGAACGGC |
| | | ACCCTGGCGCCGGGCGCGAGCGTTAGCTTCG |
| | | GTTTTAACGGTAGCGGTCCGGGTTCTCCGTCT |
| | | AACTGTAAACTGAACGGTGGTTCTTGTGATG |
| | | GTACCTCT |
| SgCBP-1 | SEQ ID NO: 17: | SEQ ID NO: 18: |
| | GSATCATAWSSSSVYTN | GGATCCGCCACCTGCGCGACCGCGTGGAGC |
| | GGTVSYNGRNYTAKW | AGCTCCAGCGTTTACACCAACGGCGGCACCG |
| | WTQNERPGTSDVWAD | TTAGCTACAACGGCCGTAACTACACCGCGAA |
| | KGACGTGGEGPGGNN | ATGGTGGACCCAGAACGAACGTCCGGGCAC |
| | GFVVSEAQFNQMFPNR | CTCTGATGTTTGGGCGGACAAAGGTGCGTGC |
| | NAFYTYKGLTDALSAY | GGCACCGGCGGCGAAGGTCCGGGTGGCAAC |
| | PAFAKTGSDEVKKREA | AACGGTTTCGTTGTTAGCGAAGCACAGTTCA |
| | AAFLANVSHETGGLFYI | ACCAGATGTTCCCGAACCGCAACGCGTTCTA |
| | KEVNEANYPHYCDTTQ | CACCTACAAAGGCCTGACCGATGCGCTGAGC |
| | SYGCPAGQAAYYGRGP | GCGTACCCGGCGTTCGCGAAAACCGGTAGCG |
| | IQLSWNFNYKAAGDAL | ATGAAGTTAAAAAACGTGAAGCGGCTGCGTT |
| | GINLLANPYLVEQDPAV | CCTGGCGAACGTGTCCCACGAAACCGGTGG |
| | AWKTGLWYWNSQNGP | CCTGTTCTACATCAAAGAAGTTAACGAAGCT |
| | GTMTPHNAIVNNAGFG | AACTACCCGCACTACTGCGATACAACCCAGT |
| | ETIRSINGALECNGGNP | CTTACGGCTGCCCGGCGGGCCAGGCGGCGTA |
| | AQVQSRINKFTQFTQIL | CTACGGTCGCGGCCCGATCCAGCTTTCTTGG |
| | GTTTGPNLSCLE | AATTTCAACTATAAAGCTGCTGGTGATGCTCT |
| | | GGGTATTAACCTGCTGGCTAACCCGTATCTGG |
| | | TTGAACAAGATCCGGCAGTTGCTTGGAAAAC |
| | | CGGTCTGTGGTATTGGAACTCTCAGAACGGT |
| | | CCGGGTACCATGACCCCGCACAACGCTATTG |
| | | TTAACAACGCTGGCTTCGGTGAAACCATTCG |
| | | TTCTATTAACGGTGCTCTGGAATGTAACGGTG |
| | | GTAACCCGGCTCAGGTTCAGTCTCGTATTAA |
| | | CAAATTCACTCAGTTCACCCAGATCCTGGGT |
| | | ACCACCACCGGTCCGAACCTGTCTTGCCTCG |
| | | AG |

TABLE 2
Primer sequences

| Protein | Primer sequence | |
|---|---|---|
| ChBD2 | F: | SEQ ID NO: 19: TCTGGATCCGGTGATTTCGTTAAACCG |
| | R: | SEQ ID NO: 20: ACACTCGAGCCACGCATCCCACAGCTGAC |
| ChBD3 | F: | SEQ ID NO: 21: TCTGGATCCGGTGATCTGGTTAAACC |
| | R: | SEQ ID NO: 22: ACACTCGAGCCAAACATCCCACAGCTGATC |
| EfCBP-1 | F: | SEQ ID NO: 23: TCTGGATCCCACGGTTACGTTGCTTC |
| | R: | SEQ ID NO: 24: ACACTCGAGGATGTTAACGACGATCG |
| PfCBP-A | F: | SEQ ID NO: 25: TCTGGATCCGGCCCGAACGCGAACCCGAT |
| | R: | SEQ ID NO: 26: ACACTCGAGGGTCGGAACAGACGCTTCAGA |
| PfCBP-B | F: | SEQ ID NO: 27: TCTGGATCCGGTCCGACCACCCCGGTT |
| | R: | SEQ ID NO: 28: ACACTCGAGGATAACCTGACCGTTGAT |
| BcCBP-1 | F: | SEQ ID NO: 29: TCTGGATCCGCCAACAACCTGGGTTCTA |
| | R: | SEQ ID NO: 30: ACACTCGAGTTTCTGCAGGCTCAGACCATCG |
| ScCBP-1 | F: | SEQ ID NO: 31: TCTGGATCCGCGACCAGCGCGACCGCGACC |
| | R. | SEQ ID NO: 32: ACACTCGAGAGAGGTACCATCACAAGAACCA |
| SgCBP-1 | F: | SEQ ID NO: 33: TCTGGATCCGCCACCTGCGCGACCGCGT |
| | R. | SEQ ID NO: 34: ACACTCGAGGCAAGACAGGTTCGGAC |

(2) Target fragment and carrier enzyme digestion: the amplified products (i.e., the target fragments) in the step (1) each are subjected to agarose gel (1%) electrophoresis and gel recovery and purification. The endonucleases BamH1 and Xho1 are used for digestion at 37° C. for 2 h to obtain enzyme digestion products. The enzyme digestion system (20 μL) is 1 μL of BamH1, 1 μL of Xho1, 2 μL of buffer, 5.5 μL (1 μg) of the target fragment, and 10.5 μL of enzyme-free water. DNA purification is carried out on the enzyme digestion products by ethanol precipitation method to obtain the purified enzyme digestion products.

(3) The purified enzyme digestion products and T4 DNA ligase are ligated at 16° C. overnight to obtain ligation products. The ligation system (20 μL) is: 1 μL (40 ng) of pET-28a(+) cloning vector, 4.2 μL (100 ng) of the purified enzyme digestion products, 0.8 μL of T4 DNA ligase, 2 μL of T4 buffer, and 12 μL of enzyme-free water.

(4) Screening of positive clones: the ligation products each are transferred into Top10 competent cells, and cultured for 16 h at 37° C. in Kanamycin-resistant Luria-Bertani (LB) medium (Kan 100 mg/mL, added with LB medium at a ratio of 1:2000). Individual colonies are selected for sequencing validation, and a bacterial solution with successful sequencing is obtained.

### Prokaryotic Expression of Target Protein

(1) 50 μL of the bacterial solution with successful sequencing is added into 5 mL of LB culture solution (added with 2.5 μL of 100 mg/mL kanamycin sulfate), cultured at 37° C. and 200 rpm for 16 h, and the extracted plasmid is obtained according to the instructions of plasmid extraction kit (Omega Bio-Tek, Inc.).

(2) 20 ng of the extracted plasmid is added into 100 μL of BL21/Rosseta competent cells, placed for 10 min, added into an electroporation cuvette (ice bath), 200 μL of LB culture solution (without antibody) is added to resuspend the cells, the cell suspension is aspirated into a 1.5 mL EP tube, and cultured at 37° C. and 200 rpm for 45 min to obtain the resuscitated cell suspension. 200 μL cell suspension is aspirated, coated on solid medium resistant to kanamycin sulfate, and cultured at 37° C. for 12 h to obtain colonies.

(3) Expanded culture of bacteria: individual colonies on the solid medium resistant to kanamycin sulfate in the step (2) are selected and cultured in 3 mL LB culture solution (resistant to kanamycin sulfate) at 37° C. and 220 rpm for 6 hours. When the optical density at 600 nm (OD₆₀₀) of the bacterial solution reaches 0.6, 2 mL of the bacterial solution is aspirated and added into 200 mL of LB culture solution (resistant to kanamycin sulfate) to continue the culture at 37° C. When the OD₆₀₀ reaches 0.5, 40 μL of 1 mol/L inducer isopropyl β-D-1-thiogalactopyranoside (IPTG) is added and cultured at 16° C. for 18 h to obtain the bacterial solution expressing the target protein.

### Purification of Target Protein

(1) The bacterial solution expressing the target protein is centrifuged at 4° C. and 4000 rpm for 30 min, the bacteria expressing the target protein are collected, 100 mL binding buffer is added to resuspend the bacteria, and the bacteria are crushed by ultrasonication in an ice-water bath (ultrasonication for 3 s, stop for 7 s, 200 cycles) to obtain a crushed mixture. The crushed mixture is centrifuged at 12000 rpm at 4° C. for 30 min after the bacteria are crushed, the supernatant is collected, and filtered with a 0.22 μm sterile filter to obtain protein supernatant.

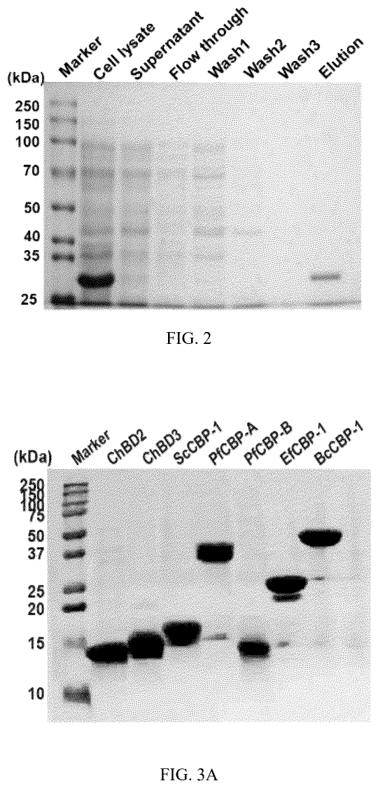

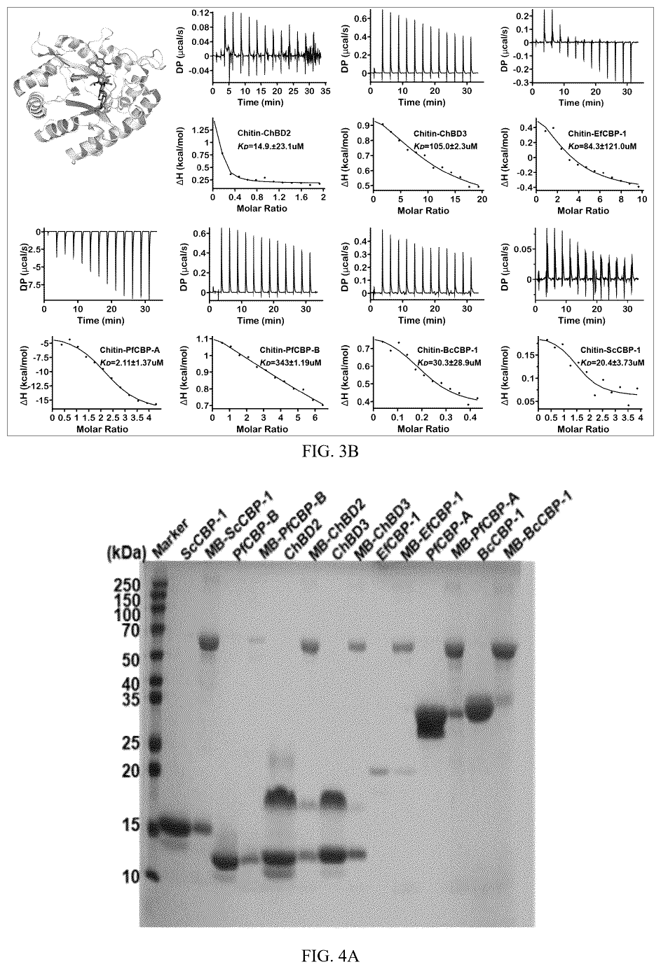

(2) Nickel ion-histidine (Ni²⁺-His) affinity chromatography: the Ni²⁺ agarose gel column is rinsed with the binding buffer, the protein supernatant in the step (1) is passed through the column (1 mL/min). After that, the column is buffered with the binding buffer, then a linear gradient of 0-100% elution buffer is set to wash the impurity protein, and the target protein is collected when the ultraviolet absorption peak (280 nm) reaches the peak. The samples of protein supernatant, washing solution and elution solution are subjected to sodium dodecyl sulfate polyacrylamide gel electrophoresis (SDS-PAGE) electrophoresis and Coomassie brilliant blue staining, and the results are shown in FIG. 2 and FIG. 3A.

(3) Concentration of target protein: the target protein in the step (2) is concentrated to 2 mL by using a centrifugal concentrator, then added with 10 mL of binding buffer, mixed well, continued to concentrate to 2 mL, repeated for 3 times, and finally concentrated to 1 mL.

(4) Determination of concentration: glycerol is added into the concentrated protein to make the final concentration 10%, in which the protein concentration determination method is BCA method, and then then marked the date and packed and stored.

FIG. 2 illustrates a purified electropherogram of a SgCBP-1 protein. As can be seen from FIG. 2, there is an obvious band of target protein in the bacteria-breaking solution (i.e., cell lysate), but there is no target protein in the supernatant channel after centrifugation. The final concentration of target protein is extremely low (<0.3 mg/mL), which is considered as insoluble inclusion body protein. The purified target protein cannot meet the requirements of subsequent experiments, so it is excluded and will not be included in the screening scope.

### Binding Affinity Between Chitin-Binding Protein and Chitin Detected by Isothermal Titration Calorimetry (ITC)

At 25° C., the ITC experiment is carried out with a MicroCal PEAQ-isothermal titration calorimeter (Malvern Instruments Ltd.) according to the ITC instruction manual. Seven proteins (ChBD2, ChBD3, EfCBP-1, PfCBP-A, PfCBP-B, BcCBP-1, ScCBP-1) and chitin are dissolved in ultrafiltration buffer. For each ITC titration, a chitin solution with a concentration of 2.0 mmol/L is titrated into a cell pool containing 0.1 mmol/L of chitin-binding protein, and titrated 13 times, with the first injection of 0.4 μL and the remaining injections of 3 μL. The data are analyzed by PEAQ-ITC analysis software, the first drop signal is removed and one set of sites fitting model is used for data analysis and fitting. The data results are shown in FIG. 3B.

It can be seen from FIGS. 3A-3B that these seven proteins have moderate affinity with chitin, and the dissociation constant K_{D}=14.9-343 μmol/L.

### Embodiment 3 Coupling of MB-CBP Complexes

Seven chitin-binding proteins (ChBD2, ChBD3, EfCBP-1, PfCBP-A, PfCBP-B, BcCBP-1, ScCBP-1) screened in the embodiment 2 are selected, and seven MB-CBP complexes (MB-ChBD2, MB-ChBD3, MB-EfCBP-1, MB-PfCBP-A, MB-PfCBP-B, MB-BcCBP-1, MB-ScCBP-1) are prepared according to the step (1) of the embodiment 1.

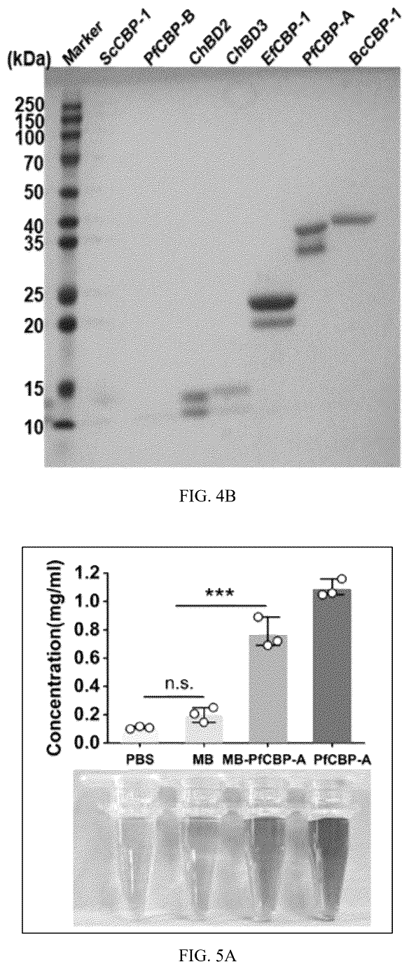

Seven MB-CBP complexes and their corresponding protein supernatants are subjected to 15% SDS-PAGE electrophoresis and Coomassie brilliant blue staining to evaluate the coupling effect, and uncoupled proteins are used as positive control. The results are shown in FIGS. 4A-4B, in which FIG. 4A illustrates the MB-CBP complexes after protein coupling by magnetic beads, and the uncoupled proteins are used as positive control, and FIG. B illustrates electrophoresis results of the corresponding supernatants of seven proteins after magnetic bead coupling.

The results show that for the same mass of activated carboxyl magnetic beads, macromolecular CBP is easier to saturate activated carboxyl magnetic beads than small molecular CBP. Therefore, the CBP with the largest molecular weight, namely PfCBP-A, is selected to determine the saturation amount of 1 mg carboxyl magnetic beads.

### Effective Particle Size Analysis

(1) Sample preparation: 10 μL of activated carboxyl magnetic beads and 10 μL of MB-PfCBP-A complex mentioned above are taken, separated the supernatant with a magnetic rack, added 3 mL of pure water containing 1% Triton-X100 for resuspension, then ultrasonicated for 15 min until the solution is clear and transparent by naked eyes and the magnetic beads move in a Brownian motion in the solvent, so as to obtain a sample to be tested, and take untreated carboxyl magnetic beads as a control.

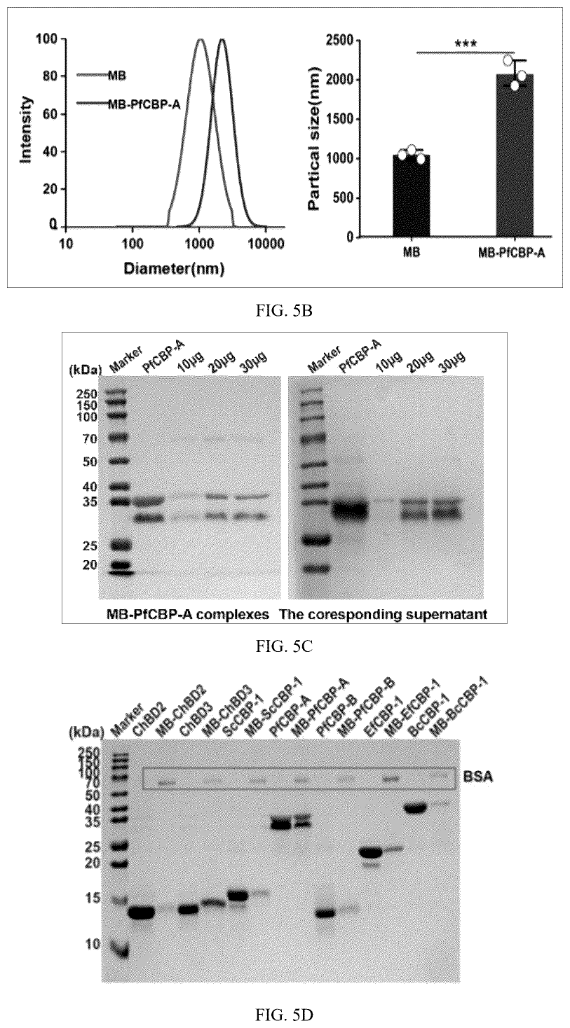

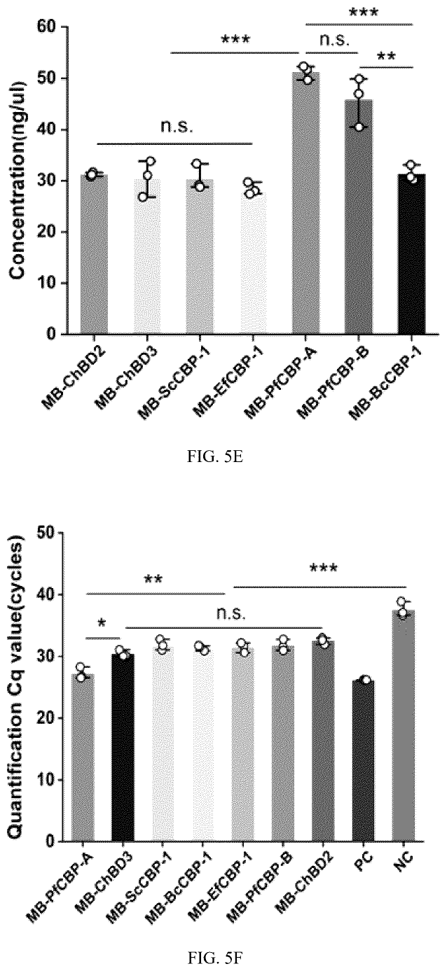

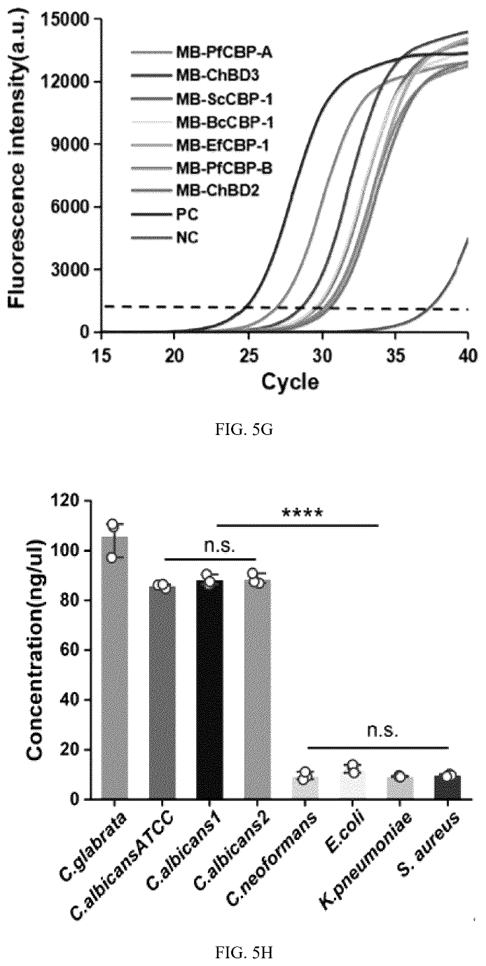

(2) NanoBrook 90PLUS PALS particle size analyzer is used, detection parameters are set as follows: angle is selected, nanoparticles are 10-250 nm, temperature is 10° C., single measurement time is 2 min, equilibrium time is 3 min, dust removal is accepted, and then the sample to be tested is loaded into the sample cell for measurement. The measurement results are shown in FIGS. 5A-5B.

The results show that compared with the control, the absorption peak of MB-PfCBP-A complex is significantly increased at A₅₆₂ₙₘ (as shown in FIG. 5A), and the determination and analysis of effective particle size show that the average effective diameter of the MB-PfCBP-A complex is twice that of the control (as shown in FIG. 5B), which proves that the PfCBP-A is successfully coupled with the activated carboxyl magnetic beads.

### Saturation Concentration Test

(1) Gradient concentrations of PfCBP-A (10 ug, 20 ug and 30 ug) are used to detect the saturating concentration of 1 mg magnetic beads. The pre-conjugated protein PfCBP-A, MB-PfCBP-A complex and corresponding supernatant are assessed by 12.5% sodium dodecyl sulfate-polyacrylamide gel electrophoresis (SDS-PAGE) followed by Coomassie blue staining. the result is shown in FIG. 5C.

It can be seen from FIG. 5C that all the coupled carboxyl magnetic beads have corresponding protein bands after electrophoresis, and the protein concentration in the separated supernatant is gradually increased. The above results further prove that the coupling of carboxyl magnetic beads and protein is successful and the minimum protein amount that can saturate 1 mg of carboxyl magnetic beads is 20 μg.

### Coupling Result Detection

In order to make all the magnetic beads coupled with seven proteins in unsaturated state, 1 mg of activated carboxyl magnetic beads is individually coupled with 15 μg each of the seven proteins (CBP), to obtain MB-CBP complexes, and then the coupling effect is evaluated by SDS-PAGE electrophoresis and Coomassie brilliant blue staining, and the corresponding CBP is used as a positive control. The results are shown in FIG. 5D.

The results show that all seven MB-CBP channels have corresponding bands, indicating that all seven proteins are successfully coupled.

### Embodiment 4 Detection of Ability of MB-CBP Complexes to Capture Candida albicans

(1) Fungal culture: 5 μL of Candida albicans preservation solution (preserved by Key Laboratory of Molecular Biology for Infectious Diseases, Ministry of Education, Chongqing Medical University) is taken and inoculated on Sabouraud Dextrose Agar (SDA) culture plate, and incubated at 37° C. for 48 h to grow a single colony. The single colony is picked in 3 mL SDA liquid culture medium, shaken at 37° C. and 220 rpm for 8 h until the OD₆₀₀ reaches 0.6, and a fungal suspension is obtained.

(2) Preparation of simulated clinical samples: the fungal suspension is centrifuged at 12000 rpm for 5 min to collect fungal cells, and the fungi are added to the blood and bronchoalveolar lavage fluid (BALF) samples (collected by Department of Laboratory Medicine, The First Affiliated Hospital of Chongqing Medical University) to prepare the simulated clinical samples.

(3) Capture and extraction of Candida albicans with MB-CBP complex: 1 mL of simulated clinical sample is taken and centrifuged at 12000 rpm for 10 min, most of the supernatant and blood cells are gently discarded, 200 μL of precipitate is remained, 50 μL of MB-CBP complex is added and incubated in a four-dimensional mixing device at 25° C. for 30 min to form a MB-CBP-C. albicans complex.

(4) The MB-CBP-C. albicans complex is gently washed for three times with 15 mmol/L PBS, and the supernatant is discarded after the washed MB-CBP-C. albicans complex is separated with the magnetic rack to obtain precipitate 1.

(5) 450 μL of recombinant cell fungal lysate (1 mol/L guanidine hydrochloride, 37.5 mL medical ethanol, 1 mL Triton-X-100, 5 mmol/L Tris-HCL, 5 mmol/L EDTA, 0.1 mg/mL protease K, pH 8.0) is added to the precipitate 1. After lysis at 55° C. for 5 min, 100 μL of 50 mg/mL silica hydroxyl magnetic beads and and 5 μL of 10 mg/mL RNase A are added and incubated at 25° C. for 10 min. The supernatant is discarded after separation with the magnetic rack to obtain the precipitate 2. 500 μL of washing buffer 1 (1 mol/L guanidine hydrochloride, 37.5 mL medical ethanol, 1 mL Triton-X-100, 10 mmol/L Tris-HCl, 10 mmol/L EDTA, pH 8.0) is added to the precipitate 2 and washed twice. 700 μL of washing buffer 2 (80% ethanol) is added and washed once. 50 μL DEPC-treated water is added into the washed precipitate 2, incubated at 55° C. for 5 min to elute genomic DNA, and separated and collected the supernatant containing DNA with the magnetic rack. The absorbance value of the supernatant of DNA is quantified at 260 nm, and the results are shown in FIG. 5E.

(6) The purity and content of genomic DNA of Candida albicans captured by the MB-CBP complex are further evaluated by using qPCR, and the results are shown in FIGS. 5F-5G.

As can be seen from FIG. 5E, seven MB-CBP complexes can effectively capture Candida albicans. Compared with other complexes, the genomic DNA concentrations of the samples captured by the MB-PfCBP-A complex and the MB-PfCBP-B complex are significantly higher than that of other complexes (P<0.001).

From FIGS. 5F-5G, it can be seen that the threshold period (Cq) of the sample captured by the MB-PfCBP-A complex is significantly lower than that of other samples. The difference between these two methods may be due to the nonspecific adsorption of the MB-PfCBP-B complex on non-target substances. Therefore, the PfCBP-A protein is identified as a candidate protein for subsequent detection.

### Embodiment 5 Detection of Capture Specificity of MB-PfCBP-A Complex

The capture specificity of MB-PfCBP-A to fungi is detected by the method described in the embodiment 4, except that in the embodiment 5, the fungal strains to be captured and isolated include: a standard strain C. albicans ATCC60193 (preserved by Key Laboratory of Molecular Biology for Infectious Diseases, Ministry of Education, Chongqing Medical University), a clinical isolated C. albicans1 (Department of Laboratory Medicine, The First Affiliated Hospital of Chongqing Medical University), a clinical isolated strain C. albicans2 (Department of Laboratory Medicine, The First Affiliated Hospital of Chongqing Medical University), a clinical isolated Candida glabrata abbreviated as C. glabrata (Department of Laboratory Medicine, The First Affiliated Hospital of Chongqing Medical University), a clinical isolated strain Clostridium neoformans abbreviated as C. neoformans (Department of Laboratory Medicine, The First Affiliated Hospital of Chongqing Medical University), a clinical isolated strain E. coli (preserved by Key Laboratory of Molecular Biology for Infectious Diseases, Ministry of Education, Chongqing Medical University), a clinical isolated strain Klebsiella pneumoniae abbreviated as K. pneumoniae (preserved by Key Laboratory of Molecular Biology for Infectious Diseases, Ministry of Education, Chongqing Medical University), and a clinical isolated strain S. aureus (preserved by Key Laboratory of Molecular Biology for Infectious Diseases, Ministry of Education, Chongqing Medical University). 10⁶ colony-forming units (CFU) of each strain is taken for capture, and after capture, the genomic DNA is extracted and purified by using lysed cells and silica hydroxyl magnetic beads, and its absorbance at A₂₆₀ₙₘ is detected. The results are shown in FIG. 5H.

The results show that the concentrations of the genomic DNA of C. albicans and C. glabrata are significantly higher than those of C. neoformans, E. coli, K. pneumoniae and S. aureus (P<0.0001). It shows that the MB-PfCBP-A complex has affinity for chitin on fungal cell wall, and can specifically capture complete fungal cells, and the C. neoformans with thick capsule and the bacterial cells without chitin cannot be recognized and captured by affinity magnetic beads.

### Embodiment 6 Clinical Verification of Affinity Molecule Detection Method

### Stability and Specificity Detection

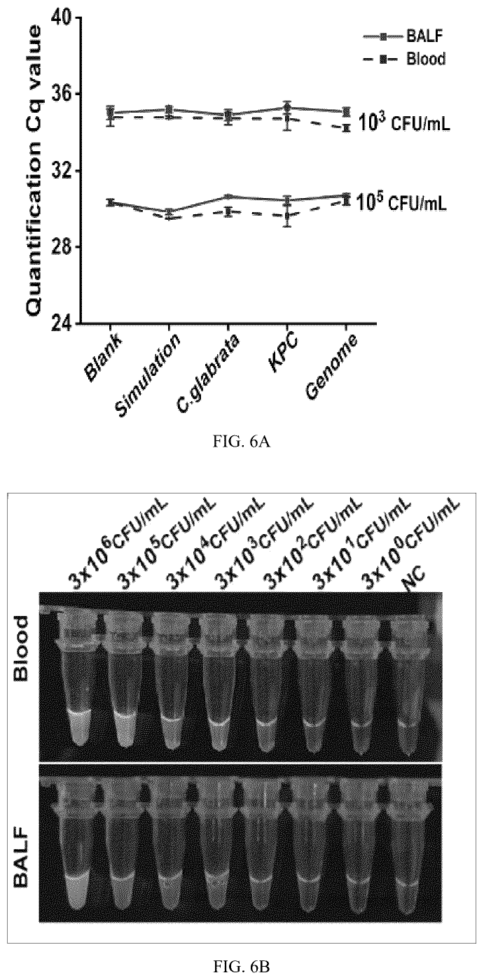

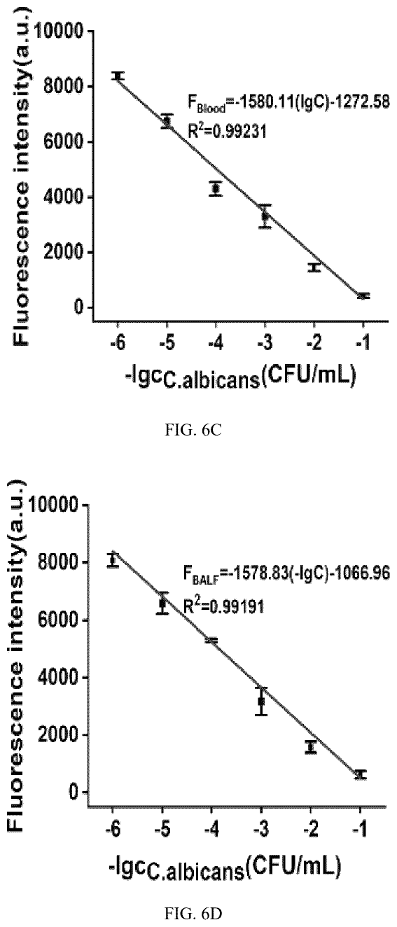

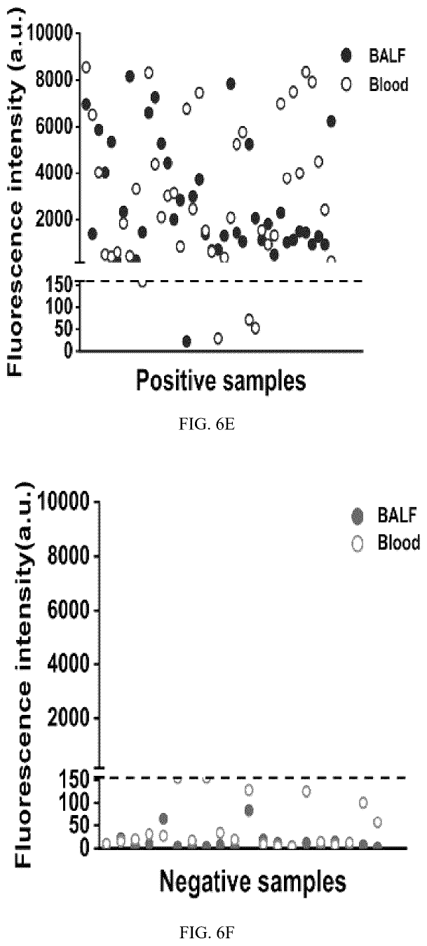

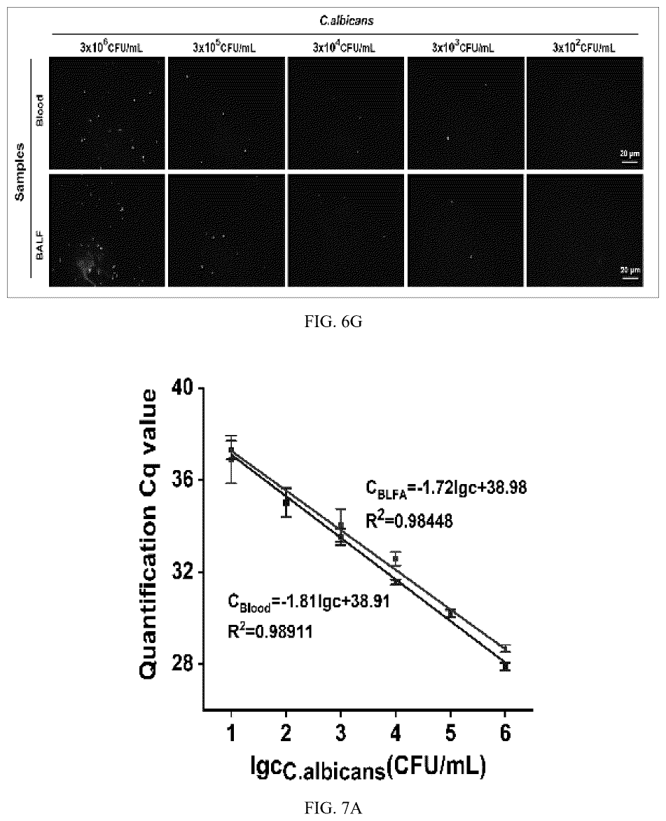

The simulated clinical samples are prepared by using healthy blood samples and bronchoalveolar lavage fluid (BALF), and the samples containing 10³ CFU/mL and 10⁵ CFU/mL C. albicans are simulated respectively. Different interfering substances, including 10³ CFU C. glabrata (collected by Department of Laboratory Medicine, The First Affiliated Hospital of Chongqing Medical University), 10³ CFU K. pneumoniae (collected by Department of Laboratory Medicine, The First Affiliated Hospital of Chongqing Medical University) and 10⁵ copies of the genomic DNA of Candida albicans, are added to the simulated C. albicans positive blood and BALF samples, respectively, 1 mL of each sample is taken for detection, and the cultured C. albicans bacterial solution is used as a blank control. The detection results are shown in FIG. 6A.

It can be seen from the FIG. 6A that the capture and separation efficiency of affinity magnetic beads is similar to that of the blank control when various interfering substances are added to the samples with low concentration (10³ CFU/mL) and high concentration (10⁵ CFU/mL). It shows that AMS technology can specifically identify and capture C. albicans, and has strong anti-interference ability against unrelated pathogens and genomes.

### Sensitivity Detection

The C. albicans ATCC60193 is added into the blood and BALF samples, and the final concentrations are 3×10⁰ CFU/mL, 3×10¹ CFU/mL, 3×10² CFU/mL, 3×10³ CFU/mL, 3×10⁴ CFU/mL, 3×10⁵ CFU/mL, and 3×10⁶ CFU/mL. The linear range of affinity molecule detection is evaluated by the detection of the affinity molecule method and the visual observation of the fluorescence signals and real-time fluorescence monitoring in the blue light. The results are shown in FIGS. 6B-6D.

As can be seen from the FIGS. 6B-6D, when the concentration of Candida albicans is between 3×10¹ CFU/mL and 3×10⁶ CFU/mL, there is an obvious linear relationship between the fluorescence intensity and the negative logarithm of the concentration of Candida albicans, with R² values of 0.99231 and 0.99191 respectively.

### Verification of Simulated Clinical Samples

Affinity molecule analysis is used to detect 80 simulated positive samples of Candida albicans (including 40 blood samples and 40 BALF samples) and 40 simulated negative samples (20 blood samples and 20 BALF samples respectively), in which the above samples are simulated clinical samples prepared by adding Candida albicans to blood and bronchoalveolar lavage fluid samples collected from The First Affiliated Hospital of Chongqing Medical University). The detection results of positive samples are shown in FIG. 6E, and the detection of negative samples is shown in FIG. 6F.

The detection cut-off value is determined by constructing the receiver operating characteristic (ROC) curve. The area under the curve (AUC) values of the blood and BALF samples are 0.976 and 0.998 respectively. Taking the fluorescence intensity corresponding to the maximum Youden index as the cut-off value, the blood and BALF samples are 156.695 and 151.276 respectively. At this time, the detection sensitivities of affinity molecule method for the blood and BALF samples are 92.5% and 97.5%, respectively, and the specificities are 100%. The sensitivity of whole blood test is 90%, while the sensitivity of BALF test is 95%. This difference may be due to the concentration of genomic DNA below the detection threshold.

### Contrast Test

At present, fungal fluorescence staining microscopy based on the reaction of chitin and chitin-binding protein is widely used in clinical samples. In this experiment, the limit of detection (LOD) between affinity molecule method and staining technique is directly compared. The detection results of the staining method are shown in FIG. 6G, and the LOD of the staining method is 3×10³ CFU/mL. This shows that the sensitivity of fungal fluorescence staining microscopy is at least 100 times lower than that of the affinity molecule method.

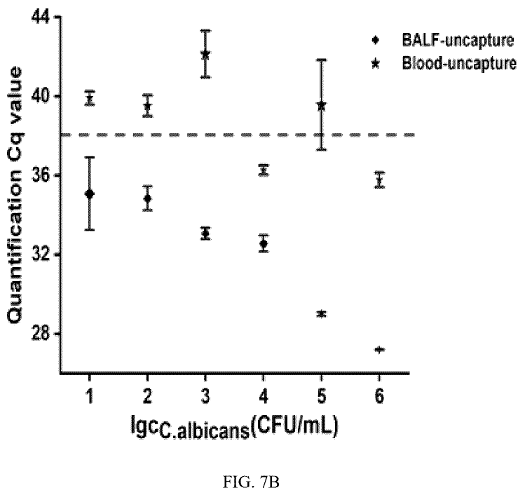

The simulated blood and BALF samples are detected by AMS-qCPR respectively, and the results are shown in FIG. 7A. The results show that the LOD of qCPR is 3×10¹ CFU/mL, and the LOD is similar to that of affinity molecule method. At the same time, 1 mL of simulated clinical samples are taken without MB-PfCBP-A capture (uncapture). After centrifugation, the cells are directly lysed, and the genomic DNA is extracted by silica hydroxyl magnetic beads and detected by qPCR. The detection results are shown in FIG. 7B, from which it is found that there is no linear relationship between the Cq value of uncaptured samples and the concentration of Candida albicans, especially the simulated blood samples, and there is no significant difference between the Cq value and negative samples.

The above experimental results show that surface affinity molecule method can capture and enrich Candida albicans from the clinical samples such as blood and bronchoalveolar lavage fluid without interference from non-target bacteria, human cells, antibodies and other substances. It emphasizes the advantages of affinity molecule method in detecting low-concentration samples, indicating that it is faster, easier to operate and more sensitive than AMS-qCPR.

As can be seen from the above embodiments, the disclosure provides the chitin-binding protein for detecting fungi and its application, and the affinity molecule detection method of fungi. The whole reaction of the detection method can be completed within 120 min, and the detection sensitivity for Candida albicans reaches 10¹ CFU/mL. The diagnosis platform is expected to provide low-cost, high-benefit, rapid, accurate and instant diagnosis for invasive fungal infections.

The above is only the illustrated embodiments of the disclosure, and it should be pointed out that those skilled in the art can make several improvements and embellishments without departing from the principle of the disclosure, and these improvements and embellishments should also be regarded as the protection scope of the disclosure.

What is claimed is:

1. A chitin-binding protein for detecting fungi, comprising at least one selected from the group consisting of ChBD2, ChBD3, EfCBP-1, PfCBP-A, PfCBP-B, BcCBP-1, and ScCBP-1;
wherein the amino acid sequence of the ChBD2 is shown in SEQ ID NO: 1, and the nucleotide sequence encoding the amino acid of the ChBD2 is shown in SEQ ID NO: 2;
wherein the amino acid sequence of the ChBD3 is shown in SEQ ID NO: 3, and the nucleotide sequence encoding the amino acid of the ChBD3 is shown in SEQ ID NO: 4;
wherein the amino acid sequence of the EfCBP-1 is shown in SEQ ID NO: 5, and the nucleotide sequence encoding the amino acid of the EfCBP-1 is shown in SEQ ID NO: 6;
wherein the amino acid sequence of the PfCBP-A is shown in SEQ ID NO: 7, and the nucleotide sequence encoding the amino acid of the PfCBP-A is shown in SEQ ID NO: 8;
wherein the amino acid sequence of the PfCBP-B is shown in SEQ ID NO: 9, and the nucleotide sequence encoding the amino acid of the PfCBP-B is shown in SEQ ID NO: 10;
wherein the amino acid sequence of the BcCBP-1 is shown in SEQ ID NO: 11, and the nucleotide sequence encoding the BcCBP-1 amino acid is shown in SEQ ID NO: 12;
wherein the amino acid sequence of the ScCBP-1 is shown in SEQ ID NO: 13, and the nucleotide sequence encoding the ScCBP-1 amino acid is shown in SEQ ID NO: 14.

2. A method using the chitin-binding protein as claimed in claim 1, comprising:
detecting the fungi by using the chitin-binding protein.

3. An affinity molecule detection method of the fungi, comprising the following steps:
step (1), performing mixed incubation on the chitin-binding protein as claimed in claim 1 and magnetic beads to obtain a magnetic bead-chitin-binding protein (MB-CBP) complex;
step (2), performing mixed incubation on the MB-CBP complex and a detection sample to obtain a mixture;
step (3), extracting a genomic DNA of the mixture; and
step (4), performing amplification and one-pot detection on the genomic DNA.

4. The affinity molecule detection method as claimed in claim 3, wherein in the step (1), a temperature of the mixed incubation is in a range of 20-30 Celsius degrees (° C.), and a time of the mixed incubation is in a range of 0.5-2.0 hours (h); and
a volume-mass ratio of the chitin-binding protein and the magnetic beads is in a range of 0.002-0.04:1.

5. The affinity molecule detection method as claimed in claim 4, wherein in the step (2), the detection sample is at least one of blood and bronchoalveolar lavage fluid.

6. The affinity molecule detection method as claimed in claim 5, wherein in the step (2), a mixed volume ratio of the MB-CBP complex and the detection sample is in a range of 0.04-0.06:0.5-1.5; and
a temperature of the mixed incubation in the step (2) is in a range of 20-30° C. and a time of the mixed incubation in the step (2) is in a range of 20-35 minutes (min).

7. The affinity molecule detection method as claimed in claim 6, wherein in the step (3), a method for the extracting the genomic DNA of the mixture is a silica hydroxyl magnetic bead method.

8. The affinity molecule detection method as claimed in claim 7, wherein in the step (4), a method for the amplification and one-pot detection of the genomic DNA is recombinase polymerase amplification-clustered regularly interspaced short palindromic repeats/CRISPR related protein 12a (RPA-CRISPR/Cas12a); and
specific steps of the RPA-CRISPR/CAS12a are as follows:
step a, performing RPA amplification on the genomic DNA and then collecting an amplified RPA system; and
step b, performing mixed incubation on the amplified RPA system and a CRISPR/Cas12a system for 5-20 min to obtain a product, and then detecting the product to judge whether there are the fungi based on intensity of a fluorescence signal.

9. The affinity molecule detection method as claimed in claim 8, wherein the CRISPR/Cas12a system comprises the following components: a 10×NE buffer2.1, a CRISPR RNA (crRNA), a Cas12a protein, and an oligonucleotide probe;
an addition amount of the 10×NE buffer2.1 is in a range of 0.1-0.6 microliters (μL);
an addition amount of the crRNA is in a range of 1-4 μL, and an initial concentration of the crRNA is 1-3 in a range of micromoles per liter (μmol/L);
an addition amount of the Cas12a protein is in a range of 0.10-1.0 μL, and an initial concentration of the Cas12a protein is in a range of 4-5 μmol/L;
an addition amount of the oligonucleotide probe is in a range of 0.10-1.5 μL, and an initial concentration of the oligonucleotide probe is in a range of 1-15 μmol/L;
the nucleotide sequence of the crRNA is shown in SEQ ID NO: 15;
the nucleotide sequence coding the Cas12a protein is shown in SEQ ID NO: 16; and
the nucleotide sequence of the oligonucleotide probe is 5′-FAM-TTATTATT-BHQ1-3′.

10. The affinity molecule detection method as claimed in claim 9, where temperature of the mixed incubation on the amplified RPA system and the CRISPR/Cas12a system in the step b is in a range of 36-44° C.
